SEQUENCE LISTING (33 sequences)

SEQ ID NO: 1 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 2 - protein, 213 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATSAEP

SEQ ID NO: 3 - protein, 209 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEAT

SEQ ID NO: 4 - protein, 233 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATKSNKYNKGLSPLLSSCHLFVAILK

SEQ ID NO: 5 - protein, 240 aa, Rattus rattus
MEKPRGVRCTNGFPERELPRPGASRPAEKSRPPEAKGAQPADAWKAGRPRSEEDNELNLPNLAAAYSSILRSLGEDPQRQGLLKTPWRAATAMQFFTKGYQETISDVLNDAIFDEDHDEMVIVKDIDMFSMCEHHLVPFVGRVHIGYLPNKQVLGLSKLARIVEIYSRRLQVQERLTKQIAVAITEALQPAGVGWIEATHMCMVMRGVQKMNSKTVTSTMLGVFREDPKTREEFLTLIRS

SEQ ID NO: 6 - protein, 240 aa, Mus musculus
MEKPRGVRCTNGFSERELPRPGASPPAEKSRPPEAKGAQPADAWKAGRHRSEEENQVNLPKLAAAYSSILLSLGEDPQRQGLLKTPWRAATAMQYFTKGYQETISDVLNDAIFDEDHDEMVIVKDIDMFSMCEHHLVPFVGRVHIGYLPNKQVLGLSKLARIVEIYSRRLQVQERLTKQIAVAITEALQPAGVGWIEATHMCMVMRGVQKMNSKTVTSTMLGVFREDPKTREEFLTLIRS

SEQ ID NO: 7 - protein, 496 aa, Homo sapiens
MPTPDATTPQAKGFRRAVSELDAKQAEAIMSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAI

SEQ ID NO: 8 - protein, 528 aa, Homo sapiens
MPTPDATTPQAKGFRRAVSELDAKQAEAIMVRGQGAPGPSLTGSPWPGTAAPAASYTPTPRSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 9 - protein, 497 aa, Homo sapiens [sequence omitted]

SEQ ID NO: 10 - protein, 501 aa, Homo sapiens [sequence omitted]

SEQ ID NO: 11 - protein, 528 aa, Homo sapiens; identical to SEQ ID NO: 8

SEQ ID NO: 12 - protein, 528 aa, Homo sapiens; identical to SEQ ID NO: 8

SEQ ID NO: 13 - protein, 498 aa, Rattus rattus
MPTPSAPSPQPKGFRRAVSEQDAKQAEAVTSPRFIGRRQSLIEDARKEREAAAAAAAAAVASSEPGNPLEAVVFEERDGNAVLNLLFSLRGTKPSSLSRAVKVFETFEAKIHHLETRPAQRPLAGSPHLEYFVRFEVPSGDLAALLSSVRRVSDDVRSAREDKVPWFPRKVSELDKCHHLVTKFDPDLDLDHPGFSDQVYRQRRKLIAEIAFQYKHGEPIPHVEYTAEEIATWKEVYVTLKGLYATHACREHLEGFQLLERYCGYREDSIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTVYWFTVEFGLCKQNGELKAYGAGLLSSYGELLHSLSEEPEVRAFDPDTAAVQPYQDQTYQPVYFVSESFNDAKDKLRNYASRIQRPFSVKFDPYTLAIDVLDSPHTIQRSLEGVQDELHTLAHALSAIS

SEQ ID NO: 14 - protein, 501 aa, Mus musculus
MSMMPTPSASSPQPKGFRRAVSEQDTKQAEAVTSPRFIGRRQSLIEDARKEREAAAAAAAAAVASAEPGNPLEAVVFEERDGNAVLNLLFSLRGTKPSSLSRALKVFETFEAKIHHLETRPAQRPLAGSPHLEYFVRFEVPSGDLAALLSSVRRVSDDVRSAREDKVPWFPRKVSELDKCHHLVTKFDPDLDLDHPGFSDQAYRQRRKLIAEIAFQYKQGEPIPHVEYTKEEIATWKEVYATLKGLYATHACREHLEAFQLLERYCGYREDSIPQLEDVSHFLKERTGFQLRPVAGLLSARDFLASLAFRVFQCTQYIRHASSPMHSPEPDCCHELLGHVPMLADRTFAQFSQDIGLASLGASDEEIEKLSTVYWFTVEFGLCKQNGELKAYGAGLLSSYGELLHSLSEEPEVRAFDPDTAAVQPYQDQTYQPVYFVSESFSDAKDKLRNYASRIQRPFSVKFDPYTLAIDVLDSPHTIRRSLEGVQDELHTLTQALSAIS

SEQ ID NO: 15 - DNA, 145 bp, adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 16 - DNA, 145 bp, adeno-associated virus 2
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 17 - DNA, 484 bp, Homo sapiens
ctagactctagctgcagagggacctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcga

SEQ ID NO: 18 - DNA, 748 bp, Homo sapiens
atggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattagga

SEQ ID NO: 19 - DNA, 155 bp, Simian virus 40
ttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgaa

SEQ ID NO: 20 - DNA, 192 bp, Homo sapiens
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 21 - DNA, 1490 bp, Simian virus 40
atgcccacccccgacgccaccacgccacaggccaagggcttccgcagggccgtgtctgagctggacgccaagcaggcagaggccatcatgtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattgg

SEQ ID NO: 22 - DNA, 610 bp, Woodchuck hepatitis B virus
cgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagct

SEQ ID NO: 23 - protein, 159 aa, Artificial Sequence
  DHFR peptide
MISLIAALAVDHVIGMENAMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 24 - protein, 159 aa, Artificial Sequence
  DHFR Y100I peptide
MISLIAALAVDHVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 25 - protein, 159 aa, Artificial Sequence
  DHFR G121V peptide
MISLIAALAVDHVIGMENAMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEVDTHFPDYEPDDWESVFSEFHDADAQNSHSYCFEILERR

SEQ ID NO: 26 - protein, 159 aa, Artificial Sequence
  DHFR F103L peptide
MISLIAALAVDHVIGMENAMPWNLPADLAWFKRNTLNKPVIMGRHTWESIGRPLPGRKNIILSSQPSTDDRVTWVKSVDEAIAACGDVPEIMVIGGGRVYEQLLPKAQKLYLTHIDAEVEGDTHFPDYEPDDWESVFSEFHDADAQNSHSYCFEILERR

SEQ ID NO: 27 - protein, 159 aa, Artificial Sequence
  DHFR N18T A19V
MISLIAALAVDHVIGMETVMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 28 - protein, 159 aa, Artificial Sequence
  DHFR H12Y/Y1001 peptide
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 29 - protein, 159 aa, Artificial Sequence
  DHFR H12L/Y1001 peptide
MISLIAALAVDLVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 30 - protein, 159 aa, Artificial Sequence
  DHFR R98H/F103S peptide
MISLIAALAVDHVIGMENAMPWNLPADLAWFKRNTLNKPVIMGRHTWESIGRPLPGRKNIILSSQPSTDDRVTWVKSVDEAIAACGDVPEIMVIGGGHVYEQSLPKAQKLYLTHIDAEVEGDTHFPDYEPDDWESVFSEFHDADAQNSHSYCFEILERR

SEQ ID NO: 31 - protein, 159 aa, Artificial Sequence
  DHFR M42T/H114R peptide
MISLIAALAVDHVIGMENAMPWNLPADLAWFKRNTLNKPVITGRHTWESIGRPLPGRKNIILSSQPSTDDRVTWVKSVDEAIAACGDVPEIMVIGGGRVYEQFLPKAQKLYLTRIDAEVEGDTHFPDYEPDDWESVFSEFHDADAQNSHSYCFEILERR

SEQ ID NO: 32 - protein, 159 aa, Artificial Sequence
  DHFR 161F/T68S peptide
MISLIAALAVDHVIGMENAMPWNLPADLAWFKRNTLNKPVIMGRHTWESIGRPLPGRKNIFLSSQPSSDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 33 - DNA, 1714 bp, Gallus gallus
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

(19) United States
(12) Patent Application Publication
(10) Pub. No.: US 2017/0114346 A1
(43) Pub. Date: Apr. 27, 2017

(54) GENE EXPRESSION SYSTEM AND REGULATION THEREOF

(71) Applicant: BRAINGENE AB, Lund (SE)

(72) Inventors: Deniz KIRIK, Lund (SE); Erik CEDERFJÄLL, Lund (SE)

(73) Assignee: BRAINGENE AB, Lund (BE)

(21) Appl. No.: 15/300,686

(22) PCT Filed: Apr. 1, 2015
(86) PCT No.: PCT/SE2015/050405

§ 371 Date: Sep. 29, 2016

(30) Foreign Application Priority Data
Apr. 3, 2014 (SE) 1450406-2

(51) Int. Cl. C12N 15/62; C12N 9/02; A61K 48/00; C12N 7/00; A61K 38/50; A61K 38/44; C12N 9/78; C12N 15/86

(52) U.S. Cl. CPC C12N 15/62; C12N 9/78; C12N 9/0071; C12Y 305/04016; C12Y 114/16002; C12N 15/86; C12N 7/00; A61K 38/50; A61K 38/44; A61K 48/005; C12N 2840/203; C12N 2750/14143

(57) ABSTRACT

The present invention relates to a novel gene expression system comprising: a) a first nucleotide sequence encoding a fusion polypeptide of: a1) a destabilizing domain (DD) based on DHFR, and a2) a GTPcyclohydrolase 1 (GCH1) polypeptide, or a biologically active fragment or variant thereof; and b) a second nucleotide sequence encoding a tyrosine hydroxylase (TH) polypeptide, or a biologically active fragment or variant thereof. The invention also relates to use of this gene expression system together with a ligand binding to a destabilizing domain (DD) based on dihydrofolate reductase (DHFR) for treatment of diseases associated with a reduced dopamine level, such as Parkinson's disease.

### FIELD OF THE INVENTION

The present invention relates to a novel gene expression system and use thereof together with a ligand, which enables control of DOPA and/or dopamine synthesis.

### BACKGROUND OF THE INVENTION

Gene therapy strategies are emerging as possible future treatment alternatives for several disease indications including applications in the brain. Recent results in clinical trials in Parkinson's disease (PD) show that such interventions are essentially safe. The animal efficacy data, e.g., for enzyme replacement by gene therapy suggests that this approach would have high probability of success in the clinics as a restorative strategy, in particular for patients with advanced disease and those that are in complication phase displaying disabling side effects of oral L-DOPA pharmacotherapy. Troublesome side effects of long term oral L-DOPA medication are thought to be a consequence of fluctuating levels of the drug and a progressive worsening of the disease.

Generation of DOPA in catecholaminergic neurons is handled by the tyrosine hydroxylase (TH) enzyme in the presence of tetrahydrobiopterin (BH₄). The inventors and others have shown that transduction of striatal neurons with transgenes encoding the TH and GTP cyclohydrolase 1 (GCH1; rate-limiting enzyme in BH₄ synthesis) enzymes with the use of adeno associated virus (AAV) vectors gives rise to continuous DOPA production in these cells (Mandel et al., 1998; Kirik et al., 2002; Carlsson et al., 2005). Reconstitution of this synthetic pathway in the striatum in turn provides the means for robust and near complete recovery from behavioral deficits seen in animal models of PD (Björklund et al., 2010).

None of the strategies implemented in animal studies or in clinical trials so far have a mechanism to regulate DA production in the brain from the transgenes delivered with viral vectors, i.e., they are irreversible and uncontrollable. It is widely acknowledged, however, that regulation of gene expression is important for long-term safety of the treatment and for the first time, it opens the possibility to truly personalize the treatment to the needs of the individual patients.

Gene regulation systems have been developed utilizing e.g., rapamycin dimerization, lac operator-repressor, different steroids receptors (ecdysone and mifepristone), RNA interference and aptamers (see Manfredsson et al., 2012 for a recent review). The most studied and better characterized regulatory system is the one that is built on the tetracycline (tet) responsive promoters. This system uses a fusion protein composed of the E. coli tet-repressor protein and the HSV-1 VP-16 transcription factor that is constitutively expressed and initiates transcription when bound to a transgenic promoter containing a tet-operon. In the presence of its inhibitory ligand tetracycline, or an analog e.g., doxycycline, the interaction of the fusion protein and the promoter sequence is inhibited (thus termed tet-off system). Many alterations of this system have been developed including versions where administration of the ligand instead promotes transcription (tet-on system).

Both tet-on and tet-off systems have been used in a number of in vivo gene therapy applications including those in the brain (see Stieger et al., 2009 for recent review). In particular, tet-dependent regulation of TH, AADC and GDNF has been reported in rodent models of PD. However, to the knowledge of the inventors this system has not been developed towards use in humans, most likely due to the concern that the expression of the HSV-1 protein in the constructs is potentially immunogenic and since majority of the adult population have circulating antibodies against this virus. Secondly, the requirements for dosing for effective gene regulation in the brain may be unfavorable as tetracycline derivatives have low blood brain barrier penetration capabilities. In addition, ligand induced toxicity can be a potential problem and has been suggested for the doxycycline, at least in cell culture experiments. Notably, despite the relatively long pre-clinical characterization period over two decades, there is still limited information available on the efficacy of the tet-operant controlled gene expression systems in non-human primate models of any brain disease.

The suitability of controlled gene therapy depends not only on the characteristics of the specific construct used but also the ligand required for controlling the transgene. Ideally, the ligand should readily cross the blood-brain barrier (BBB) and activate expression in a dose range that does not induce any unwanted effects. Furthermore, it is important that the ligand is potent enough to induce high expression for optimal therapeutic benefit in the dose range it can be applied over long-term.

The use of controlled gene expression systems would have many advantages over constitutive expression in the clinical setting. The properties of a tunable gene therapy that makes it superior to conventional uncontrolled gene therapy can readily be exemplified in the case of PD. First, individuals with PD are a heterogeneous group of patients in terms of disease severity and corresponding dopaminergic depletion. Thus dosing a single time interventional treatment that cannot be terminated or modified would pose significant concerns in the selection of the dose for each patient. A regulated system, on the other hand, would allow post-vector tailoring of the treatment efficacy based on each patient's specific needs and as a factor of the transduction extent in the target area of the brain. Thus, both in clinical development phase and during its use as a marketed drug, the capability of regulation for fine-tuning and individualizing potency of the treatment would be highly desirable. Secondly and in further support for the use of controlled gene delivery systems, PD is a progressive disorder, in which the patients' requirements for optimal therapeutic efficacy change over the course of the disease. Even if the gene therapy is carefully titrated and matched to the needs of a given patient, in the long-term, the dose chosen at the stage of the disease when the intervention is considered, will fall out of the range for optimal treatment benefits. If, on the other hand, the dose selected for the same patient were adjusted to meet the needs over a longer period of time, the initial response might lead to adverse effects, making such adjustments to initially “over-dose” the patients unlikely to be feasible in the clinics.

### SUMMARY OF THE INVENTION

Conventional symptomatic treatment for Parkinson's disease (PD) with long term Levodopa (L-DOPA) is complicated with development of troublesome drug-induced side effects. In vivo viral vector-mediated gene expression technology provides a novel drug delivery strategy with distinct advantages over pharmacotherapy. In this context, rendering striatal cells to become continuous DOPA producers (i.e., biological mini-pumps) after gene therapy using vectors containing the genes encoding tyrosine hydroxylase (TH), or a biologically active fragment or variant thereof; and GTP cyclohydrolase 1 (GCH1), or a biologically active fragment or variant thereof; eliminates fluctuations associated with oral administration of L-DOPA, which in turn results in dramatic beneficial effect in animal models of PD. Since the brain alterations made with currently employed gene transfer techniques cannot be readily re-administered and are irreversible, especially in heterogeneous diseases with slowly progressive nature, the therapeutic approaches taken to the clinic should have the possibility of regulating the expression to match the needs of each person and the stage of disease.

The inventors took advantage of a recently described tunable gene expression system based on the use of a destabilizing domain based on dihydrofolate reductase (DHFR). The inventors investigated how control of DOPA production by regulation of the two enzymes involved in DOPA synthesis could be achieved.

Due to the fact that synthesis of DOPA from tyrosine is the rate-limiting step and that the kinetics of the TH enzyme determines the efficiency of the process, a skilled person in the art would argue that regulation of DOPA synthesis in the context of gene therapy would be best accomplished by fusing the DD construct to TH protein. In other words, controlling the function of the TH enzyme by direct fusion to DD and causing destabilization due to unfolding of the TH protein should be the best choice.

On the other hand, fusion of DD to GCH1 protein would be predicted to result in a sub-optimal result as in that scenario TH enzyme, expressed constitutively, would be present in the cell at all times and any residual amount of BH4 made from low amount of GCH1 in transduced cells could readily trigger synthesis of DOPA from the TH enzyme.

Taken together, the ideal solution to the task at hand is predicted to be the combination of DD fused to TH—where the stability of the resulting enzyme is under the direct control of TMP ligand—which is co-administered with a constitutively active transgene coding for GCH1. In vitro experiments performed by the inventors showed that this combination was indeed active and regulatable.

Contrary to the anticipated solution, it was surprisingly found that in vivo a destabilizing domain based on DHFR, N-terminally fused to the GCH1 enzyme (DD-GCH1) provided far superior results as compared with fusion to the TH (DD-TH). Expression of DD-GCH1 was regulated by administration of trimethoprim (TMP)—a high affinity ligand to DHFR that crosses the blood-brain barrier. DD-GCH1 expression was combined with constitutively expressed TH enzyme to obtain the desired functional effect. The inventors show that the resulting intervention provides a TMP-dose dependent regulation of DOPA synthesis in the brain that is closely linked to the magnitude of functional effects. The data constitutes the first proof of principle for controlled reconstitution of dopamine capacity in the brain.

The present invention provides a gene expression system comprising: a first nucleotide sequence encoding a fusion polypeptide of:

a) a destabilizing domain (DD) based on DHFR, and

b) a GTP cyclohydrolase 1 (GCH1) polypeptide, or a biologically active fragment or variant thereof; and

a second nucleotide sequence encoding a tyrosine hydroxylase (TH) polypeptide, or a biologically active fragment or variant thereof.

Furthermore, the present invention relates to the above gene expression system and a ligand binding to a destabilizing domain (DD) based on dihydrofolate reductase (DHFR) for use in the treatment of a disease or condition associated with a reduced dopamine level.

Moreover, the present invention relates to a ligand binding to a destabilizing domain (DD) based on dihydrofolate reductase (DHFR) for use in the treatment of a disease or condition associated with a reduced dopamine level in a patient that previously has been subject to gene therapy whereby the above gene expression system has been administered into the brain of the patient.

The present invention further relates to a method for treatment of a disease or condition associated with a reduced dopamine level, wherein a therapeutically effective amount of the above gene expression system is administered into the brain of the patient, and wherein further a therapeutically effective amount of a ligand binding to a destabilizing domain (DD) based on dihydrofolate reductase (DHFR) is administered to the patient.

The present invention also relates to a method for controlling the DOPA and/or dopamine synthesis in the brain of a patient into whom the above gene expression system has been administered, said method comprising administration to the patient of a ligand binding to a destabilizing domain (DD) based on dihydrofolate reductase (DHFR).

The invention further relates to a conditional protein stability system comprising the above gene expression system and a ligand binding to a destabilizing domain (DD) based on dihydrofolate reductase (DHFR), wherein upon introduction of the nucleic acid sequences to a cell, the fusion polypeptide and a tyrosine hydroxylase (TH) polypeptide, or a biologically active fragment or variant thereof, are expressed, and wherein the stability of the fusion protein can be modulated by the amount of ligand present in and/or administered to the cells.

### DETAILED DESCRIPTION OF THE INVENTION

The present invention is based on the need for a controlled and/or controllable gene therapy. To achieve this goal, the inventors used a recently described tunable protein expression system based on destabilized dihydrofolate reductase (DHFR) (Iwamoto et al., 2010). Briefly, DHFR and any protein of interest coupled to it (in this case an enzyme) are readily degraded in the cell in the absence of its ligand. The addition of ligand stabilizes the protein complex, which in turn rescues the enzymatic activity leading to functional restoration in the transduced cells. It is shown below in the results that this novel protein regulation mechanism can be implemented as a gene therapy tool to control the synthesis of DOPA in the brain and secondly that the approach has robust efficacy in the rat model of PD to justify scale-up to large animal experiments and later translated to clinical studies.

Below several sequences are mentioned. These are:

SEQ ID NO 1: GTP cyclohydrolase 1 (human),

SEQ ID NO 2: GTP cyclohydrolase 1 Isoform GCH-2 (human),

SEQ ID NO 3: GTP cyclohydrolase 1 Isoform GCH-3 (human),

SEQ ID NO 4: GTP cyclohydrolase 1 Isoform GCH-4 (human),

SEQ ID NO 5: GTP cyclohydrolase 1 (rat),

SEQ ID NO 6: GTP cyclohydrolase 1 (mouse),

SEQ ID NO 7: Tyrosine 3-hydroxylase (human),

SEQ ID NO 8: Tyrosine 3-monooxygenase (human),

SEQ ID NO 9: Tyrosine hydroxylase (human),

SEQ ID NO 10: Tyrosine hydroxylase (human),

SEQ ID NO 11: Tyrosine 3-monooxygenase (human),

SEQ ID NO 12: Tyrosine 3-monooxygenase (human),

SEQ ID NO 13: Tyrosine 3-hydroxylase (rat),

SEQ ID NO 14: Tyrosine 3-hydroxylase (mouse),

SEQ ID NO 15: Adena-associated virus 2 left terminal sequence,

SEQ ID NO 16: Adena-associated virus 2 right terminal sequence,

SEQ ID NO 17: Homo sapiens synapsin 1 (SYN 1) promoter sequence,

SEQ ID NO 18: Homo sapiens GTP cyclohydrolase 1 (GCH1), transcript variant 1,

SEQ ID NO 19: Simian virus 40 early poly-adenylation sequence,

SEQ ID NO 20: Simian virus 40 late poly-adenylation sequence,

SEQ ID NO 21: Homo sapiens tyrosine hydroxylase (TH), transcript variant 2,

SEQ ID NO 22: Woodchuck hepatitis B virus (WHV8) post-transcriptional regulatory element sequence,

SEQ ID NO 23: DHFR peptide,

SEQ ID NO 24: DHFR Y1001 peptide,

SEQ ID NO 25: DHFR G121V peptide,

SEQ ID NO 26: DHFR F103L peptide,

SEQ ID NO 27: DHFR N18T A19V,

SEQ ID NO 28: DHFR H12Y/Y1001 peptide,

SEQ ID NO 29: DHFR H12L/Y1001 peptide,

SEQ ID NO 30: DHFR R98H/F103S peptide,

SEQ ID NO 31: DHFR M42T/H114R peptide,

SEQ ID NO 32: DHFR 161F/T68S peptide, and

SEQ ID NO 33: Chicken beta actin (CBA) promoter sequence.

Details on these sequences are provided in the appended sequence listing. Before the present invention is described, it is to be understood that this invention is not limited to the particular embodiments described, as such methods, and formulations may, of course, vary. Furthermore, as used herein and in the appended claims, the singular forms “a,” “an,” and “the” include plural referents unless the context clearly dictates otherwise, and includes reference to equivalent steps and methods known to those skilled in the art.

All publications mentioned herein are incorporated herein by reference to disclose and describe the specific methods and/or substances, compounds etc in connection with which the publications are cited. Unless defined otherwise, all technical and scientific terms used herein have the same meaning as commonly understood by one of ordinary skill in the art to which this invention belongs. A few exceptions, as discussed below, have been further defined within the scope of the present invention.

The invention will now be described in more detail. The embodiments described below are given as examples of the whole scope of the invention. Other solutions, uses, objectives, and functions within the scope of the invention as specified in the claims are apparent for the person skilled in the art.

The term “gene expression system”, as used herein, denotes a system specifically designed for production of a specific gene product, which in this case is at least two different polypeptides or proteins, as specified in the claims and explained in further detail below. The gene expression system may be used in vitro, but in many embodiments it is intended to be used in vivo in gene therapy.

In the context of the present application, the terms “polypeptide” and “protein” are used interchangeably. They both relate to a compound consisting of a contiguous sequence of amino acid residues linked by peptide bonds.

Furthermore, in the context of polypeptides discussed herein, the term “based on” may be used interchangeably with the term “derived from”. A polypeptide or peptide, or fragment thereof, derived from a specific polypeptide or peptide (“parent polypeptide”) has an amino acid sequence that is homologous to but not identical with the parent polypeptide. A derivative is thus a polypeptide or peptide, or fragment thereof, derived from a parent polypeptide. An analogue is such a derivative that has essentially the same function or exactly the same function as the parent polypeptide.

In the context of polypeptides discussed herein, the term “variant” may be used interchangeably with the term “derived from”. The variant may be a polypeptide having an amino acid sequence that does not occur in nature.

A mutant polypeptide or a mutated polypeptide is a polypeptide that has been designed or engineered in order to alter the properties of the parent polypeptide.

In accordance with the present invention, the first nucleotide sequence encodes a fusion polypeptide of a destabilizing domain based on DHFR and a GTP cyclohydrolase 1 (GCH1) polypeptide or a biologically active fragment or variant thereof.

The term “nucleotide sequence” as used herein may also be denoted by the term “nucleic acid sequence”. Below, the expression “gene” is sometimes used, which is also a nucleotide sequence.

The term “fusion polypeptide” as used herein relates to a polypeptide obtained after fusion, i.e. arrangement in-frame as part of the same contiguous sequence of amino acid residues. Fusion can be direct, i.e. with no additional amino acid residues between the two polypeptides, or achieved via a linker. Such a linker may be used to improve performance and/or alter the functionality.

The term “domain” used herein, which may also be denoted “region”, refers to a contiguous sequence of amino acid residues that has a specific function, such as binding to a ligand and/or conferring instability.

The first part of this fusion polypeptide comprises a destabilizing domain (DD) based on DHFR. The term “destabilizing domain of dihydrofolate reductase (DHFR)” or “DD based on DHFR” refers to a mutated variant of the wild type protein DHFR wherein the mutations have made the variant unstable. Another criterion is that the DD based on DHFR used in accordance with the invention is stabilized by the ligand used. Often the full length sequence of this is used, but it is also possible to use variants from which some amino acid residues have been deleted. The DD based on DHFR may be selected as shown by Iwamoto et al., 2010, including in the Supplemental Information to this publication.

Examples of destabilizing domains (DD) based on DHFR that may be used for the present invention are those disclosed in US 2009/0215169 A1 as SEQ ID NOS: 13-17 and 19-23, which are also included in the present disclosure as SEQ ID NOS: 23-32, as specified in the sequence listing. More precisely, in some embodiments said DD based on DHFR is at least 70% identical to a polypeptide selected from the group consisting of SEQ ID NO: 23, SEQ ID NO: 24, SEQ ID NO: 25, SEQ ID NO: 26, SEQ ID NO: 27, SEQ ID NO: 28, SEQ ID NO: 28, SEQ ID NO: 30, SEQ ID NO: 31 and SEQ ID NO: 32 as disclosed in the appended sequence listing. In some embodiments said DD based on DHFR is at least 75% identical to a polypeptide selected from the group consisting of the above mentioned sequences SEQ ID NOS: 23-32. In some embodiments said DD based on DHFR is at least 80% identical to a polypeptide selected from the group consisting of the above mentioned sequences SEQ ID NOS: 23-32. In some embodiments said DD based on DHFR is at least 85% identical to a polypeptide selected from the group consisting of the above mentioned sequences SEQ ID NOS: 23-32. In some embodiments said DD based on DHFR is at least 90% identical to a polypeptide selected from the group consisting of the above mentioned sequences SEQ ID NO: 23-32. In some embodiments said DD based on DHFR is at least 95% identical to a polypeptide selected from the group consisting of the above mentioned sequences SEQ ID NOS: 23-32. In some embodiments said DD based on DHFR is at least 96% identical to a polypeptide selected from the group consisting of the above mentioned sequences SEQ ID NOS: 23-32. In some embodiments said DD based on DHFR is at least 97% identical to a polypeptide selected from the group consisting of SEQ ID NOS. 23-32. In some embodiments said DD based on DHFR is at least 98% identical to a polypeptide selected from the group consisting of the above mentioned sequences SEQ ID NOS: 23-32. In some embodiments said DD based on DHFR is at least 99% identical to a polypeptide selected from the group consisting of the above mentioned sequences SEQ ID NOS: 23-32. In some embodiments said DD based on DHFR is 100% identical to a polypeptide selected from the group consisting of the above mentioned sequences SEQ ID NOS: 23-32.

The DD may be controlled or regulated by the use of a ligand binding and stabilizing the DD. In principle, any ligand having the stabilizing effect on the destabilizing domain as trimethoprim could be part of the embodiments according to the present invention. When this DD is fused to a second peptide, it destabilized the whole fusion polypeptide. The whole fusion polypeptide may then be controlled or regulated by the use of such a ligand, and thus also the function of the polypeptide may be controlled. Generation of controllable DHFR domains and control thereof is well-known to the skilled person, and is explained i.e. in US 2009/0215169 and Iwamoto M et al., 2010. In some embodiments, the DD based on DHFR used is a DD based on E. coli dihydrofolate reductase (ecDHFR). In some embodiments, the DD based on DHFR, such as a DD based on ecDHFR, is coupled to the N-terminal side of the GCH1 polypeptide or a biologically active fragment or variant thereof.

By fusing a destabilizing domain based on DHFR to a polypeptide, which in the present case is a GCH1 polypeptide or a biologically active fragment or variant thereof, the stability of the fusion protein, and thus in consequence also of GCH1 or fragment or variant thereof, is altered and may be affected by use of a ligand that binds to the destabilizing domain based on DHFR. The destabilizing domain based on DHFR constitutes a destabilizing domain (DD) in the fusion polypeptide. The general chemical method behind this regulation of protein stability is well-known to the skilled person, and is explained i.e. in US 2009/0215169, US 2010/0034777 and Iwamoto M et al. (2010). The stability of the fusion polypeptide can be modulated by the amount of the ligand present in the cells, and thus by the amount of the ligand administered to the patient when this system is used for in vivo gene therapy.

The second part of this fusion polypeptide comprises a GTP cyclohydrolase 1 (GCH1) polypeptide or a biologically active fragment or variant thereof. GTP cyclohydrolase 1 or GTP cyclohydrolase I, is also by the enzyme code EC 3.5.4.16, and is abbreviate herein as GCH1.

According to the invention, it is further possible to use a biologically active fragment or variant of GCH1.

In some embodiments said GCH1 polypeptide or biologically active fragment or variant thereof is at least 70% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6. In some embodiments said GCH1 polypeptide or biologically active fragment or variant thereof is at least 75% identical to a polypeptide selected from the group consisting of the above mentioned sequences SEQ ID NOS: 1-6. In some embodiments said GCH1 polypeptide or biologically active fragment or variant thereof is at least 80% identical to a polypeptide selected from the group consisting of the above mentioned sequences SEQ ID NOS: 1-6. In some embodiments said GCH 1 polypeptide or biologically active fragment or variant thereof is at least 85% identical to a polypeptide selected from the group consisting of the above mentioned sequences SEQ ID NOS: 1-6. In some embodiments said GCH 1 polypeptide or biologically active fragment or variant thereof is at least 90% identical to a polypeptide selected from the group consisting of the above mentioned sequences SEQ ID NO: 1-6. In some embodiments said GCH 1 polypeptide or biologically active fragment or variant thereof is at least 95% identical to a polypeptide selected from the group consisting of the above mentioned sequences SEQ ID NOS: 1-6. In some embodiments said GCH 1 polypeptide or biologically active fragment or variant thereof is at least 96% identical to a polypeptide selected from the group consisting of the above mentioned sequences SEQ ID NOS: 1-6. In some embodiments said GCH 1 polypeptide or biologically active fragment or variant thereof is at least 97% identical to a polypeptide selected from the group consisting of SEQ ID NOS. 1-6. In some embodiments said GCH 1 polypeptide or biologically active fragment or variant thereof is at least 98% identical to a polypeptide selected from the group consisting of the above mentioned sequences SEQ ID NOS: 1-6. In some embodiments said GCH 1 polypeptide or biologically active fragment or variant thereof is at least 99% identical to a polypeptide selected from the group consisting of the above mentioned sequences SEQ ID NOS: 1-6. In some embodiments said GCH 1 polypeptide or biologically active fragment or variant thereof is 100% identical to a polypeptide selected from the group consisting of the above mentioned sequences SEQ ID NOS: 1-6. Such GCH1 polypeptide, biologically active fragments and variants thereof have earlier been disclosed in more detail in WO 2011/054976. It is further possible to use an N-terminal truncated GCH1 polypeptide, as it is known to the skilled person that such polypeptides are biologically active (Higgins et al, 2011). It is also possible to use splice variants of a GCH1 polypeptide, as it is known to the skilled person that such polypeptides are biologically active.

In some embodiments, the part of the first nucleotide sequence that encodes the part of the fusion polypeptide consisting of a GTP cyclohydrolase 1 (GCH1) polypeptide, or a biologically active fragment or variant thereof comprises or consists of the sequence SEQ ID NO. 18.

In accordance with the present invention, the second nucleotide sequence encodes a tyrosine hydroxylase polypeptide or a biologically active fragment or variant thereof (Daubner et al, 1993; Daubner et al, 1995; Nakashima et al, 2009). The tyrosine hydroxylase, which in the present disclosure is abbreviated as TH, may also be called tyrosine 3-monooxygenase, L-tyrosine hydroxylase or tyrosine 3-hydroxylase, and is also denoted by the enzyme code EC 1.14.16.2.

According to the invention, it is further possible to use a biologically active fragment or variant of TH. This includes biologically active fragments of TH comprising at least 50 contiguous amino acids of the full length TH, wherein any amino acid specified in the selected sequence is altered to a different amino acid, provided that no more than 15 of the amino acid residues in the sequence are so altered. The biologically active fragment may be a part of the catalytic domain of tyrosine hydroxylase. The biologically active variant may be a mutated tyrosine hydroxylase polypeptide, wherein one or more of the residues S19, S31, S40 or S404 have been altered to another amino acid residue.

In some embodiments said tyrosine hydroxylase (TH) polypeptide or biologically active fragment or variant thereof is at least 65% identical to a polypeptide selected from the group consisting of SEQ ID NO: 7, SEQ ID NO: 8, SEQ ID NO: 9, SEQ ID NO: 10, SEQ ID NO: 11, SEQ ID NO: 12 SEQ ID NO: 13 and SEQ ID NO: 14. In some embodiments said TH polypeptide or biologically active fragment or variant thereof is at least 70% identical to a polypeptide selected from the group consisting of the above mentioned sequences SEQ ID NOS: 7-14. In some embodiments said TH polypeptide or biologically active fragment or variant thereof is at least 75% identical to a polypeptide selected from the group consisting of the above mentioned sequences SEQ ID NOS: 7-14. In some embodiments said TH polypeptide or biologically active fragment or variant thereof is at least 80% identical to a polypeptide selected from the group consisting of the above mentioned sequences SEQ ID NOS: 7-14. In some embodiments said TH polypeptide or biologically active fragment or variant thereof is at least 85% identical to a polypeptide selected from the group consisting of the above mentioned sequences SEQ ID NOS: 7-14. In some embodiments said TH polypeptide or biologically active fragment or variant thereof is at least 90% identical to a polypeptide selected from the group consisting of the above mentioned sequences SEQ ID NOS: 7-14. In some embodiments said TH polypeptide or biologically active fragment or variant thereof is at least 95% identical to a polypeptide selected from the group consisting of the above mentioned sequences SEQ ID NOS: 7-14. In some embodiments said TH polypeptide or biologically active fragment or variant thereof is at least 96% identical to a polypeptide selected from the group consisting of the above mentioned sequences SEQ ID NOS: 7-14. In some embodiments said TH polypeptide or biologically active fragment or variant thereof is at least 97% identical to a polypeptide selected from the group consisting of the above mentioned sequences SEQ ID NOS: 7-14. In some embodiments said TH polypeptide or biologically active fragment or variant thereof is at least 98% identical to a polypeptide selected from the group consisting of the above mentioned sequences SEQ ID NOS: 7-14. In some embodiments said TH polypeptide or biologically active fragment or variant thereof is at least 99% identical to a polypeptide selected from the group consisting of the above mentioned sequences SEQ ID NOS: 7-14. In some embodiments said TH polypeptide or biologically active fragment or variant thereof is 100% identical to a polypeptide selected from the group consisting of the above mentioned sequences SEQ ID NOS. 7-14. Such TH polypeptide, biologically active fragments and variants thereof have earlier been disclosed in more detail in WO 2011/054976, and these sequences are also provided in the appended

### SEQUENCE LISTING

In some embodiments, the second nucleotide sequence that encodes the TH polypeptide, or a biologically active fragment or variant thereof comprises or consists of the sequence SEQ ID NO. 21.

When the first and second nucleotide sequences, i.e. the nucleotide sequence encoding the fusion polypeptide of a DD based on DHFR and a GTP cyclohydrolase 1 (GCH1) polypeptide, or a biologically active fragment or variant thereof; and the nucleotide sequence encoding a TH polypeptide, or a biologically active fragment or variant thereof, are introduced into a cell, the fusion polypeptide and the TH polypeptide, or a biologically active fragment or variant thereof, are expressed. When used in an in vivo therapy, the first and second nucleotide sequences are introduced into cells in the patient. In some embodiments these cells are cells in the patient's brain, and in some embodiments these cells are cells in the patient's striatum, which is also known as the neostriatum or striate nucleus. That the nucleotide sequences are introduced into the cells includes transfection, transduction (infection) or transformation of nucleic acids into cells, such that the nucleic acids may be used by the cells to express the polypeptides.

As mentioned in the examples below, the inventors have found that the use of a fusion polypeptide of a DD based on DHFR-GCH1 and a GCH1 polypeptide, or a biologically active fragment or variant thereof results in a tight regulation of DOPA production, and these functional effects are achieved due to two levels of control; first, directly on the stability of the fusion polypeptide, resulting in control of BH4 levels, second and indirectly, stability of the TH enzyme (in addition to its biological activity) via the availability of BH4 itself, a finding that was unexpected.

By adjusting the ratio between expression of TH and the fusion polypeptide DD-GCH1 it is possible to adjust the amount of DOPA and/or dopamine that is synthetized. In some embodiments this ratio is 1:1. In some embodiments this ratio is at least 2:1. In some embodiments this ratio is at least 3:1. In some embodiments this ratio is at least 4:1. In some embodiments this ratio is at least 5:1. In some embodiments this ratio is at least 6:1. In some embodiments this ratio is at least 7:1. In some embodiments this ratio is at least 10:1. In some embodiments this ratio is 15:1. In some embodiments this ratio is 20:1. In some embodiments this ratio is 25:1. In some embodiments this ratio is 30:1. In some embodiments this ratio is 35:1. In some embodiments this ratio is 40:1. In some embodiments this ratio is 45:1. In some embodiments this ratio is 50:1.

The above mentioned ratio may be determined by measuring the activity of the expressed TH and GCH1 enzymes and/or by measuring the amount of tetrahydrobiopterin (BH₄) and/or by the amount of mRNA transcribed and/or by the amount of proteins expressed.

The desired ratio could be decided by measuring the DOPA and/or dopamine production for different ratios.

The first and second nucleotide sequences are introduced into the cells using one, two, three or further vectors. The vector/vectors used in accordance with the present invention may be a viral vector, a plasmid vector, or a synthetic vector. When two or more vectors are used they may be individually selected from this group. The vector shall be functional in mammalian cells, and in some embodiments it shall be functional in human brain cells.

When a viral vector is used, it may be selected from the group consisting of an adeno-associated vector (AAV), lentiviral vector, adenoviral vector and retroviral vector. In some embodiments it may be advantageous to use an adeno-associated vector (AAV).

For administration into cells and/or a patient, the first and second nucleotide sequences are provided in expression cassettes.

One or several expression cassette(s) as well as one or several vector(s) may be used, in accordance with well-known techniques. Normally, one or several promoters are also used.

When one vector is used, it is possible to use one expression cassette wherein the first and second nucleotide sequence (below denoted gene1 and gene2) may be arranged in the following schematic way:
promoter-gene1-IRES-gene2-pA
   or in the following alternative schematic way:
promoter-gene1-2A-gene2-pA
   or in the following alternative schematic way, based on a fusion polypeptide of gene 1 and gene 2:
promoter-[gene1-gene2]-pA

When one vector is used, it is also possible to use two expression cassettes wherein the first and second nucleotide sequence may be arranged in the following schematic way:
promoter-gene1-pA-promotor-gene2-pA.

When using one vector and two different expression cassettes, it is possible to use the vectors disclosed in WO 2011/054976.

When two vectors are used, it is possible to use one expression cassette in each vector in the following schematic way:
promoter-gene1-pA (expression cassette in first vector)
promoter-gene2-pA (expression cassette in second vector)

In the examples above, when two promoters are used they may be the same or different. Further, when two pA sequences are used they may be the same or different.

It is further possible to use three or more vectors, and one or both genes are then split into two or more vectors.

For all alternatives given above, it is possible to combine the invention with further expression cassettes and/or vectors, expressing further polypeptides.

As shown above, it is common to use one or several promoters. In some embodiments, such promoters are promoters specific for mammalian cells. In some embodiments the promoters are specific for neural cells, including mammalian neural cells. In some embodiments, the promoters are specific for promoters specific for neurons, including mammalian neurons.

In some embodiments one or all promoters is/are a constitutive promoter or a constitutively active promoter. In some embodiments, the constitutively active promoter is selected from the group consisting of CAG, CMV, human UbiC, RSV, EF-1 alpha, SV40, Mt1 and Synapsin1.

In some embodiments one or all promoters is/are an inducible promoter. In some embodiments the inducible promoter is selected from the group consisting of Tet-On, Tet-Off, Mo-MLV-L TR, Mx1, progesterone, RU486 and Rapamycin-inducible promoter.

The expression cassettes used in accordance with the present invention may further comprise a polyadenylation sequence. In some embodiments said polyadenylation sequence is a SV40 polyadenylation sequence. In some embodiments the 5′ of said polyadenylation sequence is operably linked to the 3′ of said first and/or said second nucleotide sequence.

The expression cassettes used in accordance with the present invention may further comprise a ribosomal skipping mechanism based on 2A peptides and 2A-like sequences (denoted 2A) in the above schematic illustrations) from e.g. the foot and mouth disease virus (Furler et al, 2001). The ribosomal skipping mechanism may also have other origin e.g. equine rhinitis A virus, porcine teschovirus 1 and thosea asigna virus.

The nucleotide sequences may further be operably linked to a post-transcriptional regulatory element. In some embodiments, this post-transcriptional regulatory element is a Woodchuck hepatitis virus post-transcriptional regulatory element (WPRE). In some embodiments, this Woodchuck hepatitis virus post-transcriptional regulatory element comprises or consists of the sequence SEQ ID NO. 22.

In some embodiments, the expression cassette used comprises a 5′ terminal repeat and a 3′ terminal repeat. In some embodiments this 5′ terminal repeat and a 3′ terminal repeat are selected from Inverted Terminal Repeats [ITR] and Long Terminal Repeats [LTR]. In some embodiments the 5′ and 3′ terminal repeats are AAV Inverted Terminal Repeats [ITR], and these may then comprise or consist of the sequences SEQ ID NO. 15 and/or SEQ ID NO. 16.

In some embodiments, the vector(s) used is(are) a minimally integrating vector.

The term “ligand”, as used herein, is a small molecule or functional group that binds to a polypeptide, thereby triggering a chain of events. In some embodiments the ligand is trimethoprim (TMP).

In some embodiments the ligand is an analogue or derivative of TMP that retains the function of TMP of binding to DHFR.

TMP is a well-known and routinely prescribed antibiotic with a well-documented safety profile. It can be prescribed over extended periods for prophylaxis against urinary tract infections.

In some embodiments the ligand is folic acid.

A disease or condition associated with a reduced dopamine level in accordance with the present invention is in particular a disease or conditions caused by too low levels of dopamine in the brain, and in particular in the striatum, compared to healthy subjects. Such diseases or conditions include diseases or conditions selected from the group consisting of Parkinson's disease (PD), Parkinsonism and related disorders, schizophrenia, attention deficit disorder (ADD), attention deficit hyperactivity disorder (ADHD), autism spectrum disorders, and restless legs syndrome (RLS).

In some embodiments of the invention, the disease is Parkinson's disease. The PD may be an idiopathic form of PD. The PD may also be a genetic form of PD. There are several stages of PD. Sometimes PD is divided into five different stages, and sometimes into three stages. In the context of the present invention, PD is discussed in three different stages:

Stage 1: Early stage PD or diagnosis stage, which relates to the time when someone is first experiencing symptoms and is diagnosed;

Stage 2: Manifest PD or maintenance stage, which relates to the stage when symptoms are controlled, often by medication, and

Stage 3: Advanced stage or the complex phase.

An advantage with the present invention is that all of the above three stages of PD may be treated with good results.

The treatment according to the invention consists of two major parts. The first part is administration of the gene expression system, as further discussed above, and the second part is administration of the ligand. In some embodiments, the gene expression system is administered to the patient only once, since repeated administration, in particular when using virus vectors, may cause undesirable immunological reactions. Once the gene expression system has been administered to the patient, the amount of DOPA and thus also of dopamine, synthesized by the cells into which the gene expression system has been introduced may be controlled by the amount of ligand administered to the patient. During stage 1 of PD it may be enough to administer only very small amounts of the ligand, resulting in a low but still adequate production of DOPA and/or dopamine. During stage 2 of PD the amount of the ligand may be increased to match the changing needs of the patient due to disease progression, and during stage 3, the amount can be increased further. At all stages, it is possible to maintain an adequate, well adapted treatment of the PD. Had the gene expression system not been controllable in this way, but instead always generating a certain amount of DOPA and/or dopamine, it would not have been possible to adapt the therapy to the different needs in different stages of PD.

In some embodiments the Parkinsonism, also known as Parkinson's syndrome, atypical PD or secondary PD, treated in accordance with the present invention is Parkinsonism caused by trauma, a toxin or a metabolic disease.

In some embodiments, the disorder related to PD is DOPA-responsive dystonia. In some embodiments, the disorder related to PD is multiple system atrophy.

The term treatment, as used herein, includes remediation, amelioration of a disease or condition, and the prevention of relapse of a health problem in a subject, usually following a diagnosis.

As used herein, the term “subject” includes, but is not limited to, humans, nonhuman primates such as chimpanzees and other apes and monkey species, farm animals such as cattle, sheep, pigs, goats and horses, domestic mammals such as dogs and cats, laboratory animals including rodents such as mice, rats and guinea pigs, and the like. The term does not denote a particular age or sex. Thus, adult and newborn subjects, as well as fetuses, whether male or female, are intended to be covered. In preferred embodiments, the subject is a mammal, including humans and non-human mammals. In the most preferred embodiment, the subject is a human.

The term “patient” relates to a subject that has been diagnosed with a specific disease or disorder. In some embodiments the patient is a human.

### BRIEF DESCRIPTION OF THE DRAWINGS

In the examples below, references are made to the accompanying figures on which:

FIG. 1 illustrates DOPA measurements in medium from 293 cells transfected with plasmids encoding different combinations of the DD regulated constructs. Transfection with solely TH or GCH1 or a combination of both (A). Combining constitutively expression of TH or GCH1 and with DD regulated versions of either TH and GCH1 coupled to both N- and C-terminal side of the enzymes (B or C, respectively). All conditions run as triplicates.

FIG. 2 shows striatal extracellular levels of HVA in anesthetized animals that received a regulated vector combination with DD coupled to either GCH1 (TH+DD-GCH1; n=2) or TH (DD-TH+GCH1; n=2) in a pilot microdialysis experiment. In addition, the figure shows data from animals that received the constitutively expressed vector (TH+GCH1; n=3) from the microdialysis experiment in the main study. Six baseline samples were collected before infusion of NSD-1015 via the probe for 2 h followed by two hours infusion of NSD-1015 and TMP. All animals received oral administration of TMP (2 mg/ml) prior the microdialysis experiment.

FIG. 3 illustrates the experimental design for the long-term behavioral assessment study (Experiment 2). Eighty-nine Sprague Dawley rats received 6-OHDA injections in the medial forebrain bundle and were then screened and pre-scored with amphetamine induced rotations, corridor test and stepping test. Rats were then selected based on the severity of the lesion-induced behavioral impairments and allocated into two vector treatment groups and one lesion control group. In addition, 11 rats were included in the study as intact control group. Ten weeks after the lesion, 9 animals were injected with a mixture of two vectors encoding for constitutively active TH and GCH1 genes (depicted as TH+GCH1 group), while another 19 were injected with a vector mix in which TH expression was combined with a regulated GCH1 expression (depicted as TH+DD-GCH1). All rats were then followed with repeated behavioral assessment with corridor and stepping test over a 33-week period. At 15 weeks post-AAV injection, 10 out of 19 animals in the TH+DD-GCH1 group received trimethoprim (TMP) in their drinking water. TMP was administered in three concentrations—0.5, 1.0 and 2.0 mg/ml—for 6 weeks each in a dose-escalation study design. After 33 weeks all animals were terminated and brain tissue taken for histological processing.

FIG. 4 shows the results from Experiment 1 where striatal DOPAC, HVA and 5-HIAA levels were measured in awake-freely moving animals using online microdialysis. All animals were TMP-naïve at the start of the experiment. After the baseline sampling was done over 1 hour duration (5 samples every 12 min), 20 μM TMP-lactate (dissolved in ringer solution) was administered via the probe using reverse microdialysis principle and maintained throughout the rest of the experiment. Panel A shows 1-hour average data from baseline (BL) and 6- and 12-hours after start of TMP infusion in the TH+DD-GCH1 (n=7), TH+GCH1 (n=3), Les-Sham (n=2) and intact control (n=4) groups. DOPAC levels from animals in the TH+DD-GCH1 group (n=7) are plotted individually (black lines in panel B), while the group averages of intact and lesion controls as well as the constitutively active TH+GCH1 treatment group are presented for reference (gray lines). Data traces present in panel B are floating point averages of two data points from consecutive samples plotted over a 13-hour period of observation.

FIG. 5 illustrates motor behavioral assessment in Experiment 2 of the animals that was performed using the stepping (A,B) and corridor (C) test paradigms over 33-week follow-up period in 5 phases of the experiment as indicated in the X-axis. The stepping test results are presented in the forehand (A) and the backhand direction (B). Three measurements were conducted post AAV at 6, 9 and 12 weeks before the introduction of TMP in drinking water at three doses from 0.5 mg/ml to 1 and 2 mg/ml every six weeks. During this period 2 tested were performed with 3 weeks intervals. Note that during the post-AAV assessment period, the TH+DD-GCH1 group (open circles) was an average of 19 animals, while in the TMP dose-escalation phase of the study 9 animals were followed without TMP (open circles) and 10 were followed with TMP (gray circles).

FIG. 6 illustrates regulation of transgenic GTP cyclohydrolase 1 (GCH1) fused with destabilized dihydrofolate reductase (DHFR) (depicted as DD-GCH1) in Experiment 2. Animals treated with constitutively expressed TH+GCH1 showed robust expression of transgenic human GCH1 in the striatum (B). Note that this antibody does not cross-react with rodent GCH1 and thus no immunoreactivity is observed in intact animals (A). In the absence of TMP, TH+DD-GCH1 treated rats have very faint or negative GCH1 or DHFR immunoreactivity (C,G), whereas in animals receiving TMP the immunoreactivity of GCH1, as well as DHFR, is increased to readily detectable levels (D,H). At 2 mg/ml dose level (as shown in these panels) GCH1 expression is similar to constitutive expression group (compare B and D). Scale bar represents 100 μm.

FIG. 7 shows high magnification images obtained from the striatum from animals in Experiment 2 showing part of the transduction area stained using TH immunoreactivity, cresyl violet (CV), NeuN, and glial markers Iba1 and ED1 (equivalent to human CD68) in columns from left to right. Each experimental group is represented in rows. Scale bar in Y represents 100 μm and applies to all panels.

FIG. 8 shows high magnification images obtained from the globus pallidus from animals in Experiment 2 stained using TH immunoreactivity, cresyl violet (CV), NeuN, and glial markers Iba1 and ED1 (equivalent to human CD68) in columns from left to right. Each experimental group is represented in rows. Scale bar in Y represents 100 μm and applies to all panels.

FIG. 9 contains Table 1, which shows striatal DOPA, DOPAC and HVA metabolites measured using online microdialysis in anesthetized rats from Experiment 1. Baseline (BL) levels are shown followed by measurements after 2 h infusion of NSD-1015 (AADC inhibitor) and 2 h infusion of NSD+TMP via the sampling probe. Note that the values shown are averages (SD) and the TH+DD-GCH1 group included in the study were treated with either 0.5 (n=4) or 2.0 (n=5) mg/ml TMP for 2 weeks prior sampling. Intact (n=3), Les-Sham (n=2), TH+GCH1 (n=3).

FIG. 10 shows GCH1 immunoreactivity in two non-human primates injected with AAV5-CBA-DD-GCH1 vectors. One of the monkeys was treated with TMP systemically (A-D), while the other was followed as control (E-H). Panels A and E represent injections at the highest dose tested (1.2E14 vg/ml), B and F are from areas targeted using a 3-fold diluted vector, whereas C and G are 9-fold and D and H are 27-fold dilutions of the highest dose.

### EXAMPLES

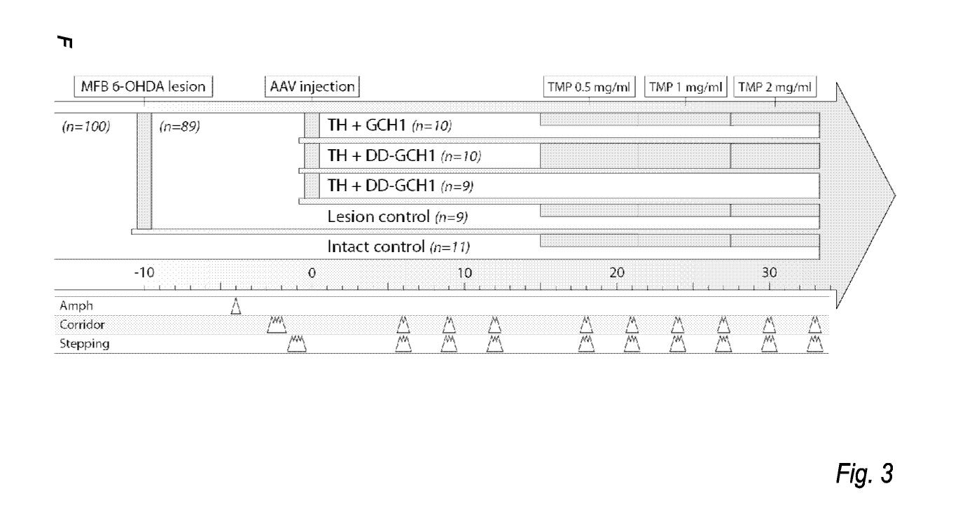

The entire study described in this document was conducted in separate parts, starting with an in vitro test followed by a pilot microdialysis experiment. Based on these results, two long-term experiments, denoted Experiment 1 and Experiment 2, were performed. Thirty-six animals were used in Experiment 1, where 4 groups of rats were subjected to an in vivo online microdialysis using one of two protocols as described below. Experiment 2 was designed to assess the long term behavioral effects of the regulated gene expression system tested in this study and included a total of 38 animals (selected from a total of 89 rats with 6-OHDA lesions) with validated severe and stable motor behavioral impairments and 11 intact control rats. The selection criteria used for inclusion in the second study was >6 net turns/min ipsilateral to the lesion side after challenge with amphetamine (2.5 mg/kg), <5% left retrievals in the corridor test, and no left forehand adjusting steps. Collectively these three measures marks animals with severe impairments induced by dopamine depletion. The details of the long-term study timeline are presented in FIG. 3.

The pilot microdialysis experiment included four rats with validated 6-OHDA lesions (>6 ipsilateral net turns/min).

### Subjects

One hundred fifty nine female Sprague-Dawley rats (Charles River, Schweinfurt, Germany) weighing 225-250 g were used in this study. Animals were housed 2-3 per cage under a 12 h light/12 h dark cycle with free access to food and water except during assessment with corridor test (as described below). All experimental procedures were approved by the Ethical Committee for use of Laboratory Animals in the Lund-Malmo region.

### Surgical Procedures

Anesthesia was induced by fentanyl citrate (Fentanyl, Apoteksbolaget, Sweden) and medetomidine hydrochloride (Dormitor, Apoteksbolaget, Sweden) injected i.p. at doses of 6 ml/kg (300 mg/kg and 0.3 mg/kg, respectively). Animals were placed in a stereotactic frame (Stoelting, Wood Dale, Ill.) and intracerebral injections were made with a Hamilton syringe (Hamilton, Bonaduz, Switzerland) fitted with a glass capillary. The anteroposterior (AP) and mediolateral (ML) coordinates were calculated from bregma and the dorsoventral (DV) coordinates from the dural surface, according to the atlas of Watson and Paxinos.

### 6-OHDA Lesions

Fourteen μg free base 6-OHDA (Sigma-Aldrich AB, Sweden) was dissolved in ascorbate-saline (0.02%), resulting in a concentration of 3.5 μg/μl, and injected into the right medial forebrain bundle using the following coordinates: AP: −4.4 mm; ML: −1.2 mm and DV: −7.8 mm with the tooth bar set to −2.4 mm. The injection speed was constant at a speed of 1 μl/min and the needle was kept in place for 3 min before it was slowly retracted.

### AAV Vector Injections

Vector preparations of TH+GCH1 or TH+DD-GCH1 were injected at two sites in the striatum with two deposits along each tract. In addition, in the pilot microdialysis experiment, a vector combination of DD-TH and GCH1 was injected in two rats with the same parameters as described here. In total 5 μl vector was injected per animal, distributed by 1.5 μl in the ventral and 1.0 μl in the dorsal deposit in each site. A pulled glass capillary (outer diameter 60-80 μm) was mounted on a Hamilton syringe with a 22-gauge needle to the minimize tissue damage and improve accuracy. The injection coordinates were: (1) AP: +1.0 mm; ML: −2.8 mm and DV: −4.5, −3.5 mm and (2) AP: 0.0 mm; ML: −4.0 mm and DV: −5.0, −4.0 mm with the tooth bar set to −2.4 mm. The injection speed was kept constant at 0.4 μl/min and the needle was kept in place for 1 min after the ventral and 3 min after the dorsal deposit. Animals in the intact and lesion control groups underwent sham surgery by drilling a burr hole at the corresponding position in the skull but without penetrating the dura.

### AAV Vectors

The viral vectors used in this study were AAV serotype 5 with ITR sequences from serotype 2, and all transgenes were driven by the chicken beta actin (CBA) promoter, which includes a rabbit gamma globulin intron and a cytomegalovirus (CMV) enhancer, and terminated with an early SV40 poly-A sequence. The two transgenes were human TH and GCH1. Regulation of GCH1 and TH expression was achieved by coupling a destabilizing domain (DD) derived from E. coli dihydrofolate reductase (DHFR) to the N- and C-terminal side of the proteins. Generation of the controllable DHFR domains has been described in detail earlier (Iwamoto et al., 2010).

Two vector combinations were used in the in vivo studies (i.e., pilot study, Experiment 1 and Experiment 2); TH and GCH1 constitutively expressed (group denoted TH+GCH1) and constitutive expression of TH combined with regulated GCH1 (DD-GCH1) (group denoted TH+DD-GCH1). All combinations were prepared in DPBS mixed at 5:1 ratio of TH or DD-TH over GCH1 or DD-GCH1. The final titers of the vectors used in Experiment 1 and Experiment 2 for TH+GCH1 and TH+DD-GCH1 were 1.9E+14 gc/ml (resulting in 9.5E11 gc injected) and 1.8E+14 gc/ml (resulting in 9.0E11 gc injected), respectively.

AAV vectors were produced in HEK-293 cells grown in tissue culture flasks for adherent cells (BD Falcon) to about 60-80% confluence. Transfection was achieved with the calcium-phosphate method and included equimolar amounts of transfer and helper plasmid DNA (pDP5 encoding for the AAV5 capsid proteins). The cells were incubated for 3 days before harvesting with PBS-EDTA. They were then centrifugated (1000×g for 5 min at 4° C.), re-suspended with lysis buffer (50 mM Tris, 150 mM NaCl, pH 8.5) and lysed by freeze-thawing cycles with dry ice/ethanol baths. The lysate was treated with benzonase (Sigma-Aldrich AB, Sweden) and then purified by centrifugation to remove cellular debris (4500×g for 20 min. at 4° C.) followed by ultracentrifugation (1.5 h at 350 000×g at 18° C.) in a discontinuous iodixanol gradient (Zolotukhin et al., 1999) and then by ion-exchange chromatography using an Acrodisc Mustang Q membrane device (Pall Life Sciences). Briefly, the Mustang Q membranes were preconditioned according to the manufacturer's instructions with a final wash with a low salt buffer (20 mM Tris, 15 mM NaCl, pH 8.0). The virus suspension was diluted threefold in the same low salt buffer, before initiating the purification. Addition of the virus to the membranes was followed by a wash with the same low salt buffer. The virus was eluted from the membranes using a high salt buffer (20 mM Tris, 250 mM NaCl, pH 8.0). The virus suspension was then buffer exchanged approximately hundredfold by adding DPBS buffer (Life technologies) and concentrated with a centrifugation filter device (Millipore Amicon Ultra 100 kDa MWCO) at 2000×g and 18° C. Dilutions of viruses were done using the same DPBS buffer. The titers of the vector preparations were determined with TaqMan quantitative PCR using primers targeting the ITR sequence promoter (Aurnhammer et al., 2012).

### In Vitro Study

HEK 293 cells were transfected with plasmids encoding DD regulated TH and GCH1, fused either on the N- or C-terminal side using Lipofectamine according to the product protocol (Life Technologies). The regulated plasmid construct was combined with either constitutively expressed TH or GCH1 in a ratio of 5:1 in favor of TH/DD-TH/TH-DD over GCH1/DD-GCH1/GCH1-DD. Six hours after transfection the culture medium was substituted with medium containing 1E-5 M TMP dissolved in 0.01% DMSO. After 24 hours, samples of the culture medium were aspirated and processed for HPLC analysis for DOPA levels. Each plasmid combination was performed as triplicates and an average was calculated.

### Oral TMP Administration

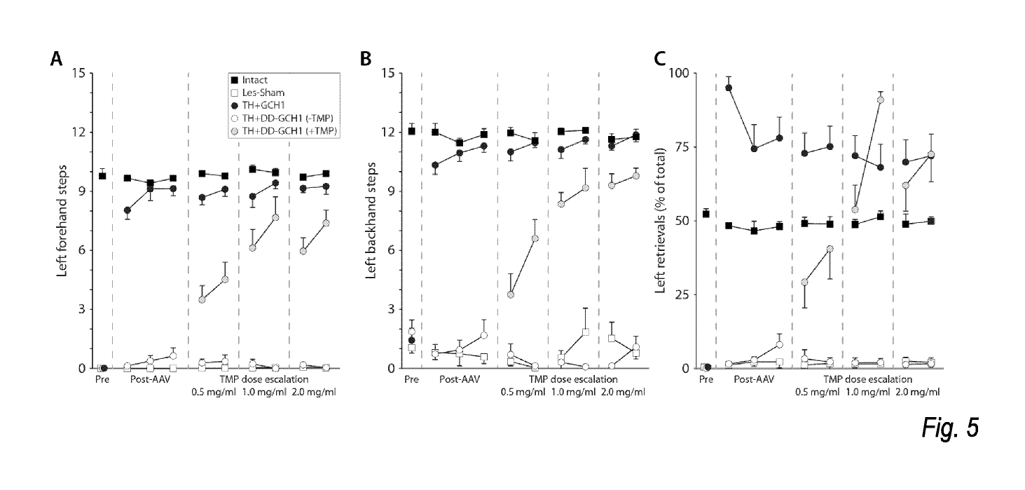

Nine of nineteen TH+DD-GCH1 treated animals in behavioral part of the experiment (Experiment 2) received oral TMP suspension (Meda AB, Solna, Sweden) in their drinking water 15 weeks post-AAV injection. TMP was administered in three different doses in 6-week intervals—starting concentration 0.5 mg/ml, 1.0 mg/ml at 21 weeks, and finally 2.0 mg/ml at 28 weeks (FIG. 5). Drinking behavior was closely monitored in a subset of animals, which enabled approximation of the corresponding dose, 22.8±2.8, 40.9±6.2, and 87.1±14.6 mg TMP/kg/day, respectively. In addition, in the microdialysis pilot all animals received 2.0 mg/ml oral TMP prior the experiment.

### Behavioral Tests

Amphetamine-induced rotation test was used as an initial screen to exclude animals with incomplete dopaminergic lesion and was performed five weeks after 6-OHDA surgeries. Animals received injections of D-amphetamine sulfate (2.5 mg/kg, i.p., Apoteksbolaget, Sweden) and their full left and right body turns were quantified over 90 minutes using automated rotometer bowls (AccuScan Instruments Inc., Columbus, Ohio). The cut-off value for net ipsilateral rotational asymmetry score was 6 full body turns/min.

Corridor test was first described by Dowd and colleagues (Dowd et al., 2005), and measures lateralized sensory neglect. Briefly, the rat was placed in the end of a corridor (150×7×23 cm) with ten adjacent pairs of cups filled with 5 sugar pellets evenly distanced along the floor of the corridor. Animals were allowed to explore the corridor freely. An investigator blinded to the group identity directly quantified retrievals; defined as each time the rat poked its nose into a unique cup, regardless of if it ate any pellets. Revisits in the same cup were not scored unless a retrieval was made from another cup in between. All rats were tested until 20 retrievals were made or the test duration exceeded 5 min. Before testing, all rats were placed in an empty corridor for 5 minutes to reduce novelty of the environment. The rats were food restricted the day prior and during the two to three days of testing. Results were calculated as an average of the contralateral retrievals (left) and presented as percentage of total retrievals.

Stepping test, developed by Schallert and colleagues (Schallert et al., 1979) and modified by Olsson et al (Olsson et al., 1995) was employed in this study. In brief, a blinded investigator assessed forelimb use by holding the rat with two hands only allowing one forepaw to touch the table surface. The investigator then moved the rat sideways over a defined distance of 90 cm with a constant speed over 4-5 sec and scored the amount of steps in both forehand and backhand direction for each forelimb. Each direction was scored twice on each testing day and the average score was calculated over 3 days.

### Online Microdialysis

In Experiment 1, two microdialysis protocols were employed in separate groups of animals. In total 36 animals were used for this part of the experiment. The first protocol was performed in four groups of TMP-naïve animals, namely the TH+DD-GCH1 (n=7), TH+GCH1 (n=4), Les-Sham (n=4) and intact controls (n=4) groups.

All animals were surgically implanted with a probe guide, which was cemented to the skull two days prior to the actual sampling. This was achieved with two screws fastened to the skull without penetrating the dura and drilling at the position of the vector injections i.e., AP: +0.5 mm; ML: −3.7 mm and DV: −1.7 mm with the tooth bar set to −2.4 mm. The DV coordinate was calculated so the membrane of the probe was positioned in the center of the transduction. A tether screw was then placed on the positioned to later hold the tether and then dental cement was added to fixate all components to the skull bone. The animal was given analgesia after the surgery and allowed to recover for at least two days before the experiment. At the day of the experiment, the animal was briefly sedated with isofluorane to easily be able to remove the guide dummy in the probe guide, insert the sampling probe and then attach the tether to the screw. The rat was placed in the testing cylinder where it had free access to food and water throughout the experiment. Following an equilibration period of 90 min baseline samples over 60 min (5 samples at 12 min intervals). The ringer solution of artificial CSF was changed to ringer containing 2E-5 M TMP lactate salt (Sigma-Aldrich AB, Sweden) so that the next sample became the first time bin when TMP was infused to the brain via reverse MD. This approach resulted in a precise measure of the time of initiation and controlled exposer of TMP over several hours following this time point. The animal was allowed to freely move in the test chamber for an additional 12 h and the dialysates were instantly injected and analyzed with a HPLC coupled to the outlet of the OMD system while the samples were collected every 12 min. The dialysates were then analyzed by HPLC with the Alexys monoamine analyzer system (Antec Leyden, The Netherlands) consisting of a DECADE II detector and VT-3 electrochemical flow cell. DA and metabolites were detected with a mobile phase consisting of 50 mM citric acid, 8 mM NaCl, 0.05 mM EDTA, 15% methanol, 700 mg/I 1-octanesulfonic acid sodium salt, at pH 3.15, with 1 mm×50 mm column with 3 mm particle size (ALF-105) at a flow rate of 90 ml/min. Peak identification and quantification was conducted using the Clarity chromatographic software package (DataApex, Prague, Czech Republic).

The second microdialysis protocol in Experiment 1 (identical microdialysis methods and protocols were used in the pilot study) was performed in anaesthetized animals was performed in TH+DD-GCH1 treated animals that received oral TMP administration in their drinking water at least 2 weeks prior sampling; either 0.5 mg/ml (n=4) or 2 mg/ml (n=5) (same TMP emulsion that was administered to the animals followed with behavior tests). In addition, this experiment also included animals from the TH+GCH1 (n=3), Les-Sham (n=2) and intact control (n=3) groups. Probe placement was calculated to position the membrane of the probe in the center of the transduction area in striatum, which corresponded to the coordinates: AP: +0.5 mm; ML: −3.7 mm and DV: −5.7 mm with the tooth bar set to −2.4 mm. After 90 min equilibration, baseline samples were collected before 1E-5 M NSD-1015 (Sigma-Aldrich, St. Louis, Mo., USA) was administered via the probe in the ringer solution for 2 h. This was then followed by a 2 h administration of 2E-5 M TMP lactate salt in addition to 1E-5 M NSD-1015 in the ringer solution. Samples were analyzed readily as described for the first microdialysis experiment. After the last sample was collected the animal was terminated and brain tissue taken for histology.

### Histological Analysis

After the last behavioral assessment point all animals were anaesthetized by an injection of 1.2 ml sodium pentobarbital (i.p., Apoteksbolaget, Sweden) and then transcardially perfused with 50 ml room temperature saline followed by 250 ml ice-cold 4% paraformaldehyde (PFA) in 0.1 M phosphate buffer adjusted to pH 7.4, at a 50 ml/min rate. The brains were then dissected and post-fixated in 4% PFA for 24 hours before cryoprotection in 25% sucrose for 24-48 hours. The fixed brains were cut in coronal orientation at a thickness of 35 μm on a semi-automated freezing microtome (Microm HM 450) and collected in 8 and 6 (striatum and substantia nigra, respectively) series and stored in anti-freeze solution (0.5 M sodium phosphate buffer, 30% glycerol and 30% ethylene glycol) at −20 litnu C.° Immunohistochemistry was performed using antibodies further processing, (AR, Rogers, Freez-Pel 1:2000 rabbit IgG, 0-40101P) raised against TH 1:2000 mouse IgG, Z3138 MCA) 1GCHAbD Serotec, Oxford, UK), AADC (AB1569, rabbit IgG, 1:500, Millipore, Billerica, Mass.), NeuN (MAB377, mouse IgG 1:500, Millipore), IBA1 (019-19741, rabbit IgG 1:1000, Wako, Richmond, Va.), ED1 (MCA341-R, mouse IgG 1:200, Serotec, Oxford, UK), and DHFR (custom made, Rabbit IgG, 1:50 000). Incubation with biotinylated secondary antibodies (BA1000, goat anti-rabbit 1:200 and BA2001, horse anti-mouse 1:200, Vector Laboratories, Burlingame, Calif.) was followed by a second 1-hour incubation with avidin-biotin peroxidase solution (ABC Elite, Vector Laboratories, Burlingame, Calif.). The staining was visualized using 3,3′-diaminobenzidine in 0.01% H₂02.

### Primate Studies

### Animals and Housing

All animal studies were conducted according to the European (EU Directive 86/609/EEC) and the French regulations (authorization n° A 92-032-02). The animal facilities are authorized by local veterinarian authorities and comply with Standards for Humane Care and Use of Laboratory Animals of the Office of Laboratory Animal Welfare (OLAW—n^{o}#A5826-01) for CEA laboratories.

Experiments were conducted on 2 male cynomolgus monkeys (Macaca fascicularis) supplied by Noveprim (Mauritius Island) of 4 and 6 years of age and weighing 5.8 and 3.8 kg. Experimental protocols and appropriate animal care procedures were authorized by special Decrees of the French. All efforts were made to minimize animal suffering and animal care was supervised by veterinarians and animal technicians skilled in the healthcare and housing of non-human primates. All NHPs were housed under standard environmental conditions (12-hour light-dark cycle, temperature: 22±1° C. and humidity: 50%) with free access to food and water. NHPs received diet (containing less than 1% folate per 10 kg of food) from the beginning of the experimental protocol.

### The MPTP Model of PD

Parkinsonism was induced by systemic administration of 1-methyl-4-phenyl-1,2,3,6-tetrahydropyridine, MPTP (Sigma, St Louis, Mo., USA) as previously described (Aron Badin et al, 2013). Briefly, non-human primates (NHPs) were exposed to daily intramuscular injections of 0.25 mg/kg MPTP for 7 consecutive days and cycles of MPTP intoxication were repeated with MPTP-free washout periods between cycles until a stable parkinsonian state was achieved. NHPs were scored daily on a scale of 0-14 according to relevant clinical scales used in PD patients and primates looking at posture, dystonia, tremor, and akinesia (Papa & Chase, 1996; Obeso et al, 2000). Parkinsonism was considered satisfactory based on the clinical scores and the presence of significant and stable reduction in spontaneous locomotor activity (by at least 80% compared to baseline) that lasted at least one month.

### Imaging Studies

Magnetic resonance imaging (MRI)

MRI was performed on all NHPs shortly before or after the baseline PET scan in order to allow precise determination of regions of interest for PET analysis and the coordinates for surgical delivery of viral constructs.

NHPs were anesthetized with 10:1 mg/kg ketamine:xylazine and placed in the magnet in a sphinx position, fixed by mouth and ear bars to a stereotactic MRI-compatible frame (M2E, France). Once in the magnet, NHPs were heated by a hot air flux and their temperature and respiration parameters monitored remotely.

MRI was performed on a 7 Tesla horizontal system (Varian-Agilent Technologies, USA) equipped with a gradient coil reaching 100 mT/m (300 μs rise time) and a circular radiofrequency 1H coil (12 cm inner diameter). T2-weighted images were acquired using a fast spin-echo sequence with the following parameters: TR=4750 ms, effective TE=62 ms, acquisition time=16 min, FOV=115×115 mm and matrix=256×256 resulting in a 450×450 μm in plane resolution, 40 coronal slices, slice thickness=1 mm. T2*-weighted images were acquired using a multiple gradient echo sequence with the following parameters: TR=2000 ms, effective TE=20 ms, acquisition time=8 min30 s, flip angle=40°, with identical geometric parameters as the T2-weighted images.

Total scan duration: 35 minutes.

### Surgery

### Surgically-Placed Gastrostomy (SPG)

In the interest of refining the procedure of daily oral delivery of the antibiotics and in order to minimize the stress induced by this manipulation, the NHP receiving TMP was equipped with a subcutaneous chamber connected to the stomach with a small catheter. The SPG device, consists of an injection port (X-Port, BARD Access Systems, France) with a self-sealing silicone septum (base 22.6 mm×28.2 mm, internal volume 0.6 ml) with attachable 8 Fr. Groshong® radio-opaque silicon catheter (50 cm long, internal diameter 1.5 mm, volume 0.6 ml). In particular, a Groshong valve, positioned at the end of the catheter, helps to prevent gastric juice reflux into the port/catheter system. All materials are biocompatible. Only non-coring needles were used (22 gauge, 2.5 cm long, BARD Access Systems, France) to puncture the silicone septum of the port, minimizing the risk of damaging it (Fante et al, 2012).

Anesthesia was induced by intramuscular injections of 10:1 mg/kg ketamine:xylazine and maintained under propofol (1 mg/kg/hour) throughout the procedure. An oral antibiotic treatment was administered before surgery (amoxicillin and clavulanic acid, 45 mg/kg/daily and 6 mg/kg/daily) and repeated daily for 5 post-operative days. Following a midline substernal laparotomy (about 3 cm long), a subcutaneous pouch was fashioned on the upper left side of the abdomen, then in the left antero-lateral site of the rib cage where the port was to be placed. The anterior wall of the stomach was identified and exteriorized. Using the Seldinger technique, the catheter was inserted in the gastric lumen through its anterior face, between the gastric body and the antrum, equidistant and 7-8 cm from the lesser and greater gastric curvatures. Then the catheter was anchored to the gastric wall with a purse-string suture (resorbable Vicryl 3/0) and passed through the left muscle layers of the anterior abdominal wall, 1-2 cm from the costal arch and about 2 cm from the midline incision. Pexy between the stomach and the abdominal wall around the catheter exit site was performed with 4 resorbable Vicryl 3/0 stitches. The catheter was connected to the port that was then inserted subcutaneously in the thoracic subcutaneous pouch and anchored to the external fascia of the rib cage (resorbable Vicryl 3/0), enabling a stable attachment and good usability when the port needle was used. The port was tested and the midline was sutured in a double layer (single suture, resorbable Vicryl 2/0).

### Intracerebral Microinjections

NHPs were induced by intramuscular injections of 10:1 mg/kg ketamine:xylazine and maintained anesthetised with propofol (1 mg/kg/hour) throughout the procedure. NHPs were placed in a dedicated MRI-compatible stereotactic frame with the head resting on a mouth bar, fixed by blunt ear bars. Temperature was maintained at 37° C. using a feed-back coupled heating blanket, and the respiratory rate, pO2, pCO2, cardiac rhythm and blood pressure were continuously monitored. All injections were performed using a dedicated Hamilton syringe and a 26G sterile needle. A midline incision was performed on the head and skin and muscle were retracted in order to access the skull. A surgical drill (point 0.280, 30000 rμm) was used to open 8 holes through the skull without piercing the dura matter. Baseline MRI images were used to calculate all injection targets in the caudate (AC+1, AC+4) and the (AC & AC-4) and 20 μL of virus were delivered per site in a single deposit at a rate of 1 μL/minute using an injection micropump (KDS30, France). Each NHP received bilateral intra-striatal injections of an AAV5-CBA-DD-GCH1 (add 1.2E14 vg/ml).

Each caudate and putamen was injected twice with the same concentration of virus. The caudate nucleus in the left hemisphere received the highest concentration and 3-fold dilution was used for the putamen on the same side, whereas the right hemisphere was injected with two 9 and 27-fold dilutions in the caudate and putamen, respectively. NHPs were housed in A2 biosafety level facilities for 3 weeks following viral injection.

Blood and CSF were collected on the day of surgery and at euthanasia in order to evaluate the presence of viral antigens.

### TMP Administration and Monitoring

TMP administration begun at 1 month post-injection, which allowed for the viral vector to reach high expression level in the different brain regions targeted. TMP was administered to only one of the two NHPs using the SPG device. TMP was administered daily for 2 months at a constant dose of 20 mg/kg. After administration of the appropriate dose, the catheter and chamber were rinsed with 10 ml of distilled water. The treated primate was weighted weekly to adjust the dose if necessary. TMP was kept in the dark at room temperature throughout the experiment.

Blood samples were collected on the day of surgery and at 1 and 2 months after TMP administration in order to measure folates and TMP. All NHPs were anesthetized 1.5 hours after TMP administration in order to avoid interfering with the absorption of the TMP molecule. TMP levels in blood were measured by liquid chromatography with UV detection (Phatophy, France).

### Euthanasia & Post-Mortem Studies

Before euthanasia NHPs were deeply anesthetized and blood and CSF were collected 1.5 h after TMP administration in the case of one primate. At the end of the experimental protocol, monkeys were euthanized by a lethal dose of pentobarbital delivered before transcardial perfusion with ice-cold 0.9% NaCl. The brains were extracted and placed in a dedicated NHP brain matrix on ice (M2E, France) bearing 2 mm subdivisions in the antero-posterior axis of the caudate and putamen in order to extract punches for biochemistry on certain brain slices and for immunohistochemistry in other slices.

Two blocks were subdivided into three 2 mm-thick slices that were placed on a petri dish on ice to obtain punches of 3 mm Ø. Samples were weighed and immediately frozen on dry ice.

All brains were post-fixed for 4 days in 4% paraformaldehyde and then cryo-preserved by immersion into sucrose-containing phosphate buffer gradients with increasing concentrations (5-10-20%) for 3 days at a time. Brains were then sliced into 40 μm-thick slices and floating slices were stained with an antibody against GTP cyclohydrolase 1 enzyme, as detailed elsewhere in this document.

### Statistical Analysis

In FIG. 5, non-parametric statistics were performed with Mann-Whitney U for between group comparisons and Friedman's test followed by Wilcoxin signed ranks test to assess the effect of TMP dose. Statistical comparisons were conducted with the SPSS statistical package version 21 (SPSS Inc., Chicago, Ill.).

### Results

### Reconstitution of DOPA, Dopamine Metabolites by the Regulated Gene Expression System

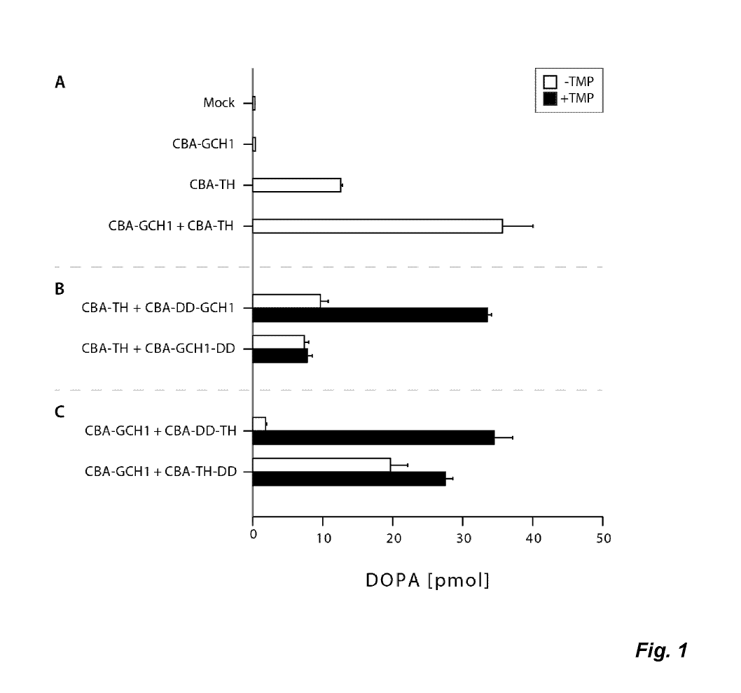

The inventors designed the first part of the study to test different plasmid combinations in an in vitro setting to determine the function, capacity and basal activity of the DD regulation system when fused with TH and GCH1 genes. For this purpose, they studied N- and C-terminal fusion peptides of the two enzymes. The readout measurement was DOPA levels in the culture medium from 293 cells transfected with the different plasmid combinations of the various constructs tested (FIG. 1). Transfection with plasmids constitutively expressing GCH1 enzyme alone did not result in any measurable DOPA production as were the mock transfected cells. Expression of TH resulted in low but detectable DOPA production compared to when both TH and GCH1 enzymes were expressed in the cells suggesting that in this cell line optimal TH activity required additional GCH1 activity that needed to be supplied exogenously (FIG. 1A). Combining constitutively expressed TH and DD-GCH1 resulted in efficient DOPA production when the DD was coupled on the N-terminal side of the enzyme and in the presence of TMP. In the absence of TMP, DOPA levels were similar to what were observed in TH only transfected cells. Coupling the DD to the C-terminally to GCH1 was detrimental since this construct did not support enhanced DOPA synthesis (FIG. 1B). Regulation of TH by coupling the DD on the N-terminal side gave DOPA levels comparable to the constitutively expressed vector combination in presence of TMP (C). Coupling DD to the C-terminal side of TH appeared not to be regulatable as it resulted in high levels of DOPA in both the presence and absence of TMP. These in vitro results suggested that combining DD on the N-terminal side was the most favorable placement and the lowest basal activity and leakage was achieved by controlling the TH enzyme.

Notably DD-TH combined with constitutively expressed GCH1 appeared to be working well, as in the presence of TMP, DOPA levels were at the same level as TH+GCH1 combination showing that in vitro this system displayed the full dynamic range as would be predicted based on earlier results reported in literature prior to performing the experiments.

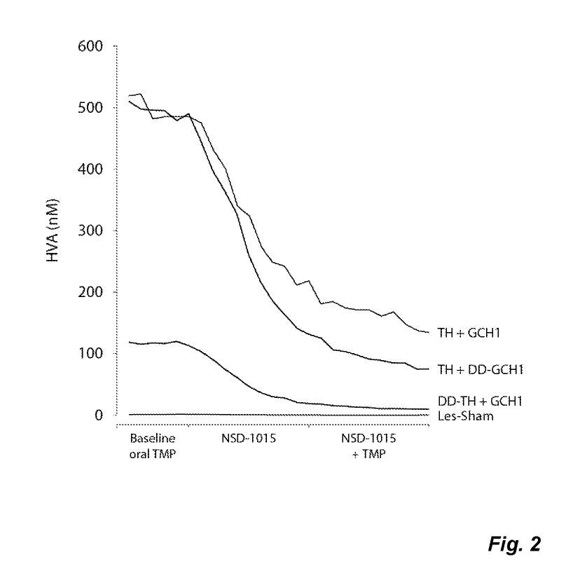

Next, the functionality of the TH and GCH1 N-terminally coupled to DD was tested in an in vivo microdialysis experiment (FIG. 2). First, animals injected with DD-TH+GCH1 were tested with online microdialysis (OMD) to measure the vector-derived DA metabolites in the brain. These animals received oral TMP emulsion in their drinking water prior to the OMD measurements. The experiment was conducted to compare the efficacy and dynamic range of the TMP controlled gene expression systems as compared with the constitutive constructs (i.e., TH+GCH1). Surprisingly, we found that contrary to observations in cell lines, and the results that would have been anticipated by skilled person, treatment with the DD-TH+GCH1 vector combination resulted in 5-fold lower levels of HVA, as compared with the constitutively active vectors. Next, we tested whether TH combined with DD-GCH1 would have the same limitation as the DD-TH+GCH1 combination. Unexpectedly, the inventors found that the DD-mediated control of GCH1 stability and therefore the availability of BH4 was very effective. This was exemplified in two ways: First the production of DOPA from DD-GCH1 in the presence of TMP was as high as the condition when constitutively expressed genes are used. Secondly, in the absence of TMP, the DOPA levels were not different from the appropriate control group suggesting that the behaviour of this system in the brain was different from the a priori anticipated outcome. It was both tightly controlled and had the full dynamic. The inventors designed two long-term in vivo studies, denoted below as Experiment 1 and Experiment 2.

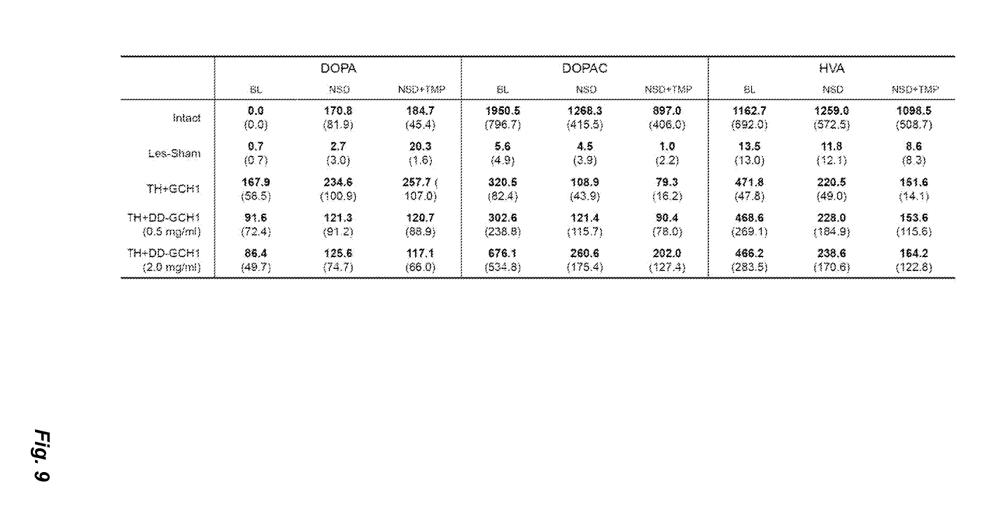

Experiment 1 was designed to validate the functionality of controlled DOPA synthesis in the striatum obtained by gene therapy incorporating a destabilized domain based on DHFR (DD) coupled to GCH1 gene in combination with constitutively expressed TH (TH+DD-GCH1). For this purpose, the inventors used two online microdialysis (OMD) study protocols. First, in anesthetized rats they determined (1) steady state production of DOPA, DOPAC and HVA under baseline conditions; (2) total DOPA synthesis capacity after inhibition of AADC in the striatum of animals where stabilization of the GCH1 protein was induced with oral administration of TMP, thus permitting synthesis of BH₄ that can activate the TH enzyme (Table 1 in FIG. 9). In intact rats, DOPAC and HVA levels in the extracellular space were abundant (typically between 1 and 2 μM), while DOPA concentrations were below reliable quantification limit of the system (2 nM). In rats with complete 6-OHDA lesion of the ascending dopaminergic projections of the medial forebrain bundle, the DOPAC and HVA concentrations fell to 1.0-25.8 nM, representing more than 99% depletion on the average. In animals treated with the TH+GCH1 constitutively active vectors, both DOPAC and HVA levels were increased (to values about 15-40% of intact controls. Notably, there was a readily detectable DOPA present in the extracellular space. Similarly, in the group treated with TH+DD-GCH1 and maintained under TMP activation, the DOPA, DOPAC and HVA levels were increased above the lesion baseline, although the DOPA concentrations appeared lower than the constitutively active group.

In order to explore the maximum capacity of the enzymatic machinery obtained from transgenic expression of the human TH enzyme, the second protocol followed the initial baseline period with addition of NSD-1015 to block the AADC enzyme. Consequently, the levels of DOPAC and HVA declined while the DOPA accumulated in all vector treated animals, suggesting that the newly synthesized DOPA was continuously converted to DA by the endogenously present residual AADC enzyme and contribute to generation of DOPAC and HVA measured in the samples obtained during the OMD experiment. Notably, the results obtained with addition of TMP to the ringer solution used as the dialysate did not further increase the DOPA levels suggesting that the system was fully activated with the oral TMP given to the animals during the 2 weeks prior to the OMD experiment. Finally, the inventors have tested two oral TMP doses (0.5 and 2.0 mg/ml in drinking water) and found that both were effective in reaching a similar biochemical reconstitution under these experimental conditions.

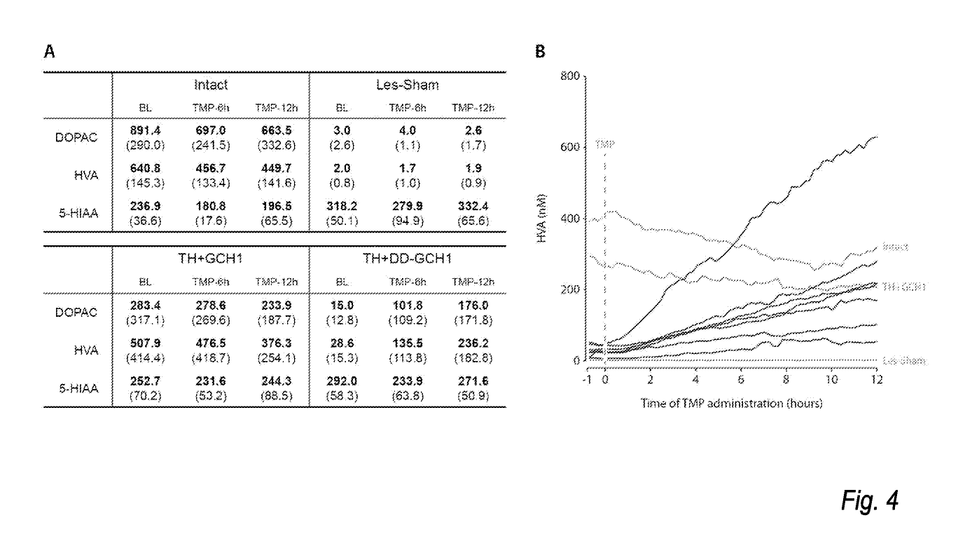

After establishing that the controlled expression system had comparable efficacy in the steady state on long-term TMP administration, the inventors turned their attention to the transition from a baseline state in the absence of TMP to the activated state upon introduction of the ligand. To be able to perform an OMD analysis in an analogous way, while anticipating a longer sampling period, the inventors adapted the measurements to be carried out in awake and freely moving animals and monitored the dialysis samples over 15-17 hours continuously and quantified levels of DOPAC, HVA and as a control also 5HIAA, the metabolite of serotonin (data from 13 hours sampling presented in FIG. 4A). As in the first protocol performed in anesthetized rats, DOPAC and HVA levels were severely depleted in the complete 6-OHDA lesioned group accounting for about 0.5% of values compared with intact controls. In rats treated with the AAV vector mix expressing the TH and GCH1 proteins constitutively (TH+GCH1 group), the DOPAC and HVA levels were about 30% and 80% of intact baseline, respectively. Notably, the TH+DD-GCH1 group (naïve to TMP) had very low baseline levels of both DOPAC and HVA, corresponding to about 2 to 4% of normal values and slightly higher when compared with lesion controls. Addition of 20 μM TMP in the dialysis solution resulted in gradual accumulation of DOPAC and HVA that at the 12 h mark were comparable to the constitutive expression group, i.e., reached to approximately 25 and 50% of normal values in intact animals as compared with about 70 and 60% in the TH+GCH1 group, respectively (FIG. 4A). Interestingly, the animals in the TH+DD-GCH1 group varied from each other in that 4 of 7 showed HVA levels within the same range of constitutive TH+GCH1 and intact groups at the 12 h point, while 2 cases had a lower value and one was about 2-fold higher than the intact average value (FIG. 4B).

Collectively, these data established that DD-GCH1 mediated controlled DOPA production worked best in this widely used animal model of PD with equal efficiency to constitutively active constructs and far better than the results obtained with the DD-TH vector.

### Behavioral Recovery in TH+DD-GCH1 Treated Rats in Response to Oral Administration of TMP

In Experiment 2, the inventors assessed long-term motor recovery in lesioned animals treated with active vectors using the stepping and corridor tests and compared them to lesion and intact controls (See FIG. 3 for experimental design). Intact naïve rats (n=1; black squares in FIG. 5) perform on average 9-11 steps in the forehand direction and 11-13 steps in the backhand direction in the stepping test (FIG. 5A,B). The normal performance in the corridor test is equal number of nose pokes in lids (retrievals) positioned on the left and right side of the corridor (FIG. 5C). The 6-OHDA lesioned rats (n=9; open squares in FIG. 5) used in this study have a severe impairment in all three parameters; i.e., make few or no adjusting steps and have a near complete bias to taking pellets from the lid on the right side, ipsilateral to the lesion. Treatment with TH+GCH1 (n=10; black circles in FIG. 5) resulted in near complete recovery in the stepping test already 6 weeks post-AAV and this effect was maintained throughout the rest of the study (FIG. 5A,B). In the corridor test, animals in this group showed an initial overcompensation at the 6-week assessment point where they almost exclusively made left retrievals (FIG. 5C). This effect was decreased at the following assessment time points but the animals continued to display an approximate 75% left side bias for the remaining time of the study until 33 weeks post-AAV injection.

Animals that received the TH+DD-GCH1 (n=19) vector showed a very limited or no recovery during the baseline assessment at 6, 9 and 12 weeks (open circles in FIG. 5). During this assessment period, the inventors observed that this group behaved similar to lesion controls and their own baseline measurements prior to vector treatment suggesting that in the absence of TMP, i.e., when GCH1 enzyme is destabilized and presumably BH₄ levels are low. This was in contrast to the constitutively active TH+GCH1 group that showed clear signs of functional changes in both behavioral tests. The follow-up period of 3 months allowed us to demonstrate that the DD-regulated delivery system was tightly controlled and could be maintained without any functional consequences to the animals over long-term.

Fifteen weeks post-AAV, half of the animals in this group (n=10; gray circles in FIG. 5) started receiving oral TMP in their drinking water while the remaining rats (n=9; open circles in the TMP dose escalation phase) continued receiving drinking water without TMP. Three concentrations of TMP were administered to the animals in escalating doses between 0.5, 1 and 2 mg/ml, each lasting 6 weeks. Based on this concentration and the amount of water consumed per day, the TMP dose per rat was estimated to be 22.8±2.8, 40.9±6.2 and 87.1±14.6 mg TMP/kg/day, respectively. Behavioral assessment was performed at three and six weeks with each TMP dose. During the 0.5 mg/ml TMP administration phase TH+DD-GCH1 group showed partial recovery already at the first assessment point at 3 weeks, in contrast to animals that received the same vector but no TMP or lesion controls (p<0.001 in all three parameters tested). When the animals received a TMP dose of 1 and 2 mg/ml the therapeutic effect in the stepping test increased gradually to a magnitude comparable to the TH+GCH1 group and were not different from intact controls in corridor test. Notably, at the second assessment of the 1 mg/ml TMP dose, an overcompensation effect was seen in the corridor test (approximately 90% retrievals on the left, previously neglected side) similar to the observations in the TH+GCH1 group at 6 weeks after transduction (FIG. 5C). This effect was transient and was not observed in the following test 3 weeks later, despite the fact that the dose of TMP was increased to 2 mg/ml.

### Oral TMP Administration Stabilizes DHFR-Coupled GCH1 and Rescues TH Expression

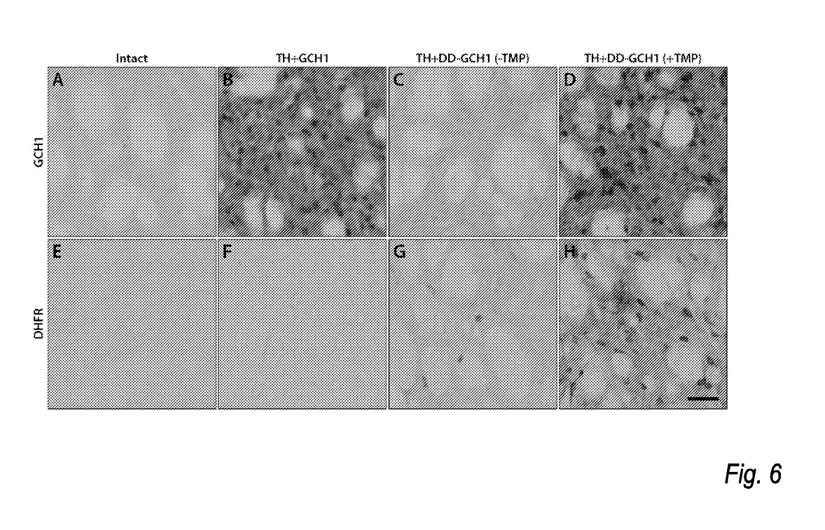

After the last behavioral assessment, brain tissue from all animals was processed for histological analysis. The expression of the GCH1 transgene was documented using an antibody specific to the human protein (with no cross reactivity to the rodent species; see lack of staining in the untreated side, FIG. 6A). As expected this analysis showed that there was a robust expression in both the constitutively active TH+GCH1 group (FIG. 6B) and the TH+DD-GCH1 group receiving the TMP ligand (FIG. 6D), while those animals that were maintained without TMP showed minimal or no immunoreactivity to GCH1 protein (FIG. 6C). Using an antibody directed to the DD, the inventors were able to confirm that the GCH1 immunoreactivity in the TH+DD-GCH1 group was accompanied with a matching staining for the DHFR (FIG. 6H) and that the same antibody stained only few cells in the group that did not receive TMP (FIG. 6G).

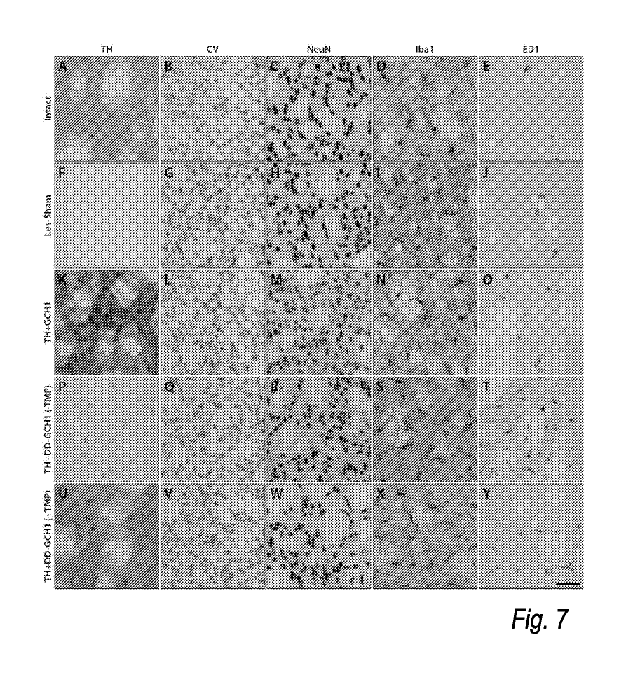

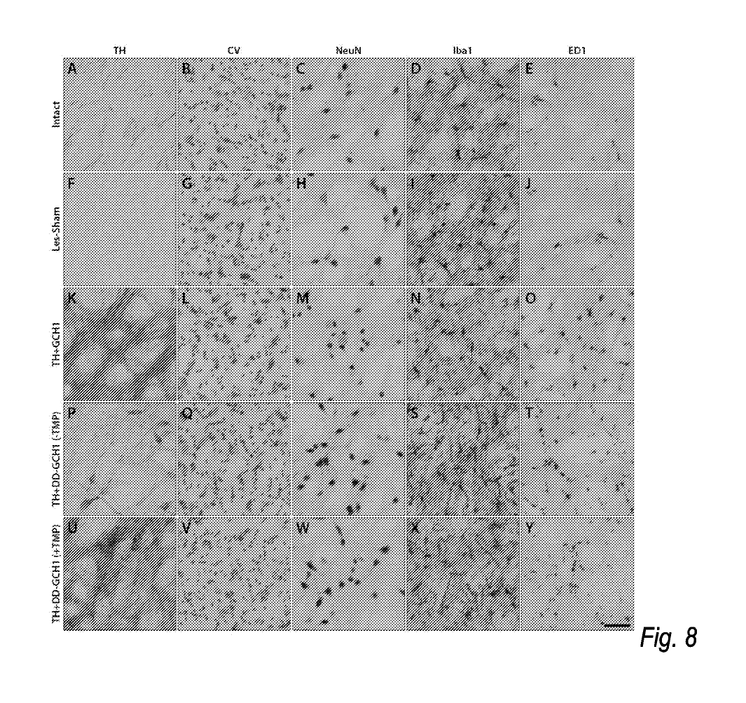

With respect to the expression of the TH enzyme, 6-OHDA lesion removes the endogenous dopaminergic fiber terminals in the striatum essentially completely (compare FIG. 7A with F), which can also be appreciated as loss pre-terminal axon projections seen transiting the globus pallidus (GP) (compare FIG. 8A with F). Expression of the human TH transgene from the AAV vector introduces a new TH enzyme pool within the striatal neurons (FIG. 7K) as well as the GP (FIG. 8K). Interestingly in the TH+DD-GCH1 group that did not receive TMP, the absence of GCH1 immunoreactivity was associated with low level of TH expression observed only in a few cells (FIG. 7P and FIG. 8P). This was in contrast to animals in the TH+DD-GCH1 group that received TMP, which showed similarly robust TH expression that were seen in animals in the constitutively active TH+GCH1 group (compare panels K and U in FIGS. 7 and 8).

These observations suggested that the tight regulation of DOPA production and functional effects from DD-GCH1 vector was achieved due to two levels of control; first, directly on the stability of the DD-fused CGH1 enzyme, resulting in control of BH₄ levels, second and indirectly, stability of the TH enzyme (in addition to its biological activity) via the availability of BH₄ itself.

Finally, the inventors assessed any potential toxic effects of transgene expression by staining serial sections from all groups of animals with either CV to see all cellular profiles, NeuN to assess the total neuronal profiles, Iba1 and ED1 antibodies for evidence of microglial activation. CV and NeuN stained specimens at the level of striatum and GP (second and third columns in FIGS. 7 and 8) showed no clear evidence of cell loss or perivascular hypercellularity and no apparent alterations were seen in NeuN-positive profiles either. No clear alterations in microglial morphologies were noted in the Iba1 stained specimens and abundance of ED1 profiles were only minimally increased suggesting that the activation of the neuroinflammatory processes were of low grade (compare groups within column 4 and 5 in FIGS. 7 and 8).

### Translation of the Tunable Gene Expression Concept to Non Human Primates

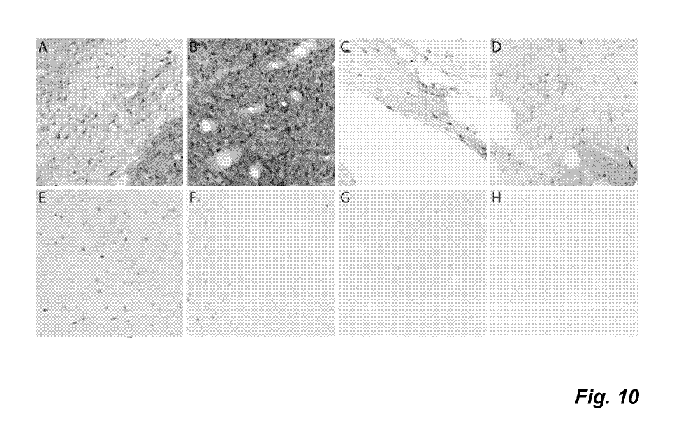

The studies in rodents were followed up with a proof-of-concept experiment in a non-human primate model of Parkinson's disease induced by systemic MPTP intoxication. Once the stabel parkinsonian state was established, two monkeys were dosed with AAV5-CBA-DD-GCH1 vectors at 4 increasing doses between (5.0E12 and 1.2E14 vg/ml) in each of the two sides of the brain using caudate nucleus and putamen as separate sites for injections. One of the monkeys was then treated with TMP to stabilize the DD-GCH1 fusion protein, while the other one was maintained without any TMP. At the end of the followup period, both monkeys were killed and tissue processed for histological documentation of gene expression in the brain. FIG. 10 shows the GCH1 immunoreactive cells in the caudate nucleus and putamen. Panels A-D display activation of the transgene in the target nuclei. The strong induction of immunoreactivity after TMP treatment in this brain shows that GCH1 protein expression can be controlled in the non-human primate brain via systemically administered TMP. In the absence of TMP the residual staining is seen only with the highest dose and in small number of cells, while in the three other doses there is essentially no/minimal specific staining detected.

### REFERENCES

Aron Badin R, Spinnewyn B, Gaillard M C, Jan C, Malgorn C, Van Camp N, Dollé F, Guillermier M, Boulet S, Bertrand A, Savasta M, Auguet M, Brouillet E, Chabrier P E, Hantraye P. IRC-082451, a novel multitargeting molecule, reduces L-DOPA-induced dyskinesias in MPTP Parkinsonian primates. PLoS One. 2013; 8(1):e52680. doi: 10.1371/journal.pone.0052680.
Aurnhammer C, Haase M, Muether N, Hausl M, Rauschhuber C, Huber I, Nitschko H, Busch U, Sing A, Ehrhardt A, Baiker A (2012) Universal real-time PCR for the detection and quantification of adeno-associated virus serotype 2-derived inverted terminal repeat sequences. Human gene therapy methods 23:18-28.
Banaszynski L A, Chen L-C, Maynard-Smith L A, Ooi A G L, Wandless T J (2006) A rapid, reversible, and tunable method to regulate protein function in living cells using synthetic small molecules. In: Cell, pp 995-1004.
Björklund T, Carlsson T, Cederfjall E A, Carta M, Kirik D (2010) Optimized adeno-associated viral vector-mediated striatal DOPA delivery restores sensorimotor function and prevents dyskinesias in a model of advanced Parkinson's disease. In: Brain, pp 496-511.
Carlsson T, Winkler C, Burger C, Muzyczka N, Mandel R J, Cenci A, Björklund A, Kirik D (2005) Reversal of dyskinesias in an animal model of Parkinson's disease by continuous L-DOPA delivery using rAAV vectors. In: Brain, pp 559-569.
Corti O, Sanchez-Capelo A, Colin P, Hanoun N, Hamon M, Mallet J (1999) Long-term doxycycline-controlled expression of human tyrosine hydroxylase after direct adenovirus-mediated gene transfer to a rat model of Parkinson's disease. In: Proc Natl Acad Sci USA, pp 12120-12125.
Daubner S C, Lohse D L, Fitzpatrick P F, (1993) Expression and characterization of catalytic and regulatory domains of rat tyrosine hydroxylase. In: Protein Sci vol. 2 (9) pp. 1452-60
Daubner S C, Piper M M, (1995) Deletion mutants of tyrosine hydroxylase identify a region critical for heparin binding. In: Protein Sci vol. 4 (3) pp. 538-41
Dowd E, Monville C, Torres E M, Dunnett S B (2005) The Corridor Task: a simple test of lateralised response selection sensitive to unilateral dopamine deafferentation and graft-derived dopamine replacement in the striatum. In: Brain Res Bull, pp 24-30.
Furler S, Paterna J C, Weibel M, Bieler H, (2001) Recombinant AAV vectors containing the foot and mouth disease virus 2A sequence confer efficient bicistronic gene expression in cultured cells and rat substantia nigra neurons. In: Gene Ther vol. 8 (11) pp. 864-73
Higgins C E, Gross S S. The N-terminal peptide of mammalian GTP cyclohydrolase I is an autoinhibitory control element and contributes to binding the allosteric regulatory protein GFRP. J Biol Chem. 2011 Apr. 8; 286(14):11919-28.
Iwamoto M, Björklund T, Lundberg C, Kirik D, Wandless T J (2010) A general chemical method to regulate protein stability in the mammalian central nervous system. In: Chem Biol, pp 981-988.
Kirik D, Georgievska B, Burger C, Winkler C, Muzyczka N, Mandel RJ, Björklund A (2002) Reversal of motor impairments in parkinsonian rats by continuous intrastriatal delivery of L-dopa using rAAV-mediated gene transfer. In: Proc Natl Acad Sci USA, pp 4708-4713.
Mandel R J, Rendahl K G, Spratt S K, Snyder R O, Cohen L K, Leff S E (1998) Characterization of intrastriatal recombinant adeno-associated virus-mediated gene transfer of human tyrosine hydroxylase and human GTP-cyclohydrolase I in a rat model of Parkinson's disease. In: J Neurosci, pp 4271-4284.
Manfredsson F P, Bloom D C, Mandel R J (2012) Regulated protein expression for in vivo gene therapy for neurological disorders: progress, strategies, and issues. Neurobiology of disease 48:212-221.
Nakashima A, Hayashi N, Kaneko Y S, Mori K, Sabban E L, Nagatsu T, Ota A, (2009) Role of N-terminus of tyrosine hydroxylase in the biosynthesis of catecholamines. In: J Neural Transm vol. 116 (11) pp. 1355-62
Obeso, J. A., Rodriguez-Oroz, M. C., Rodriguez, M., DeLong, M. R., and Olanow, C. W. 2000. Pathophysiology of levodopa-induced dyskinesias in Parkinson's disease: problems with the current model. Ann Neurol 47:S22-32; discussion S32-24.
Olsson M, Nikkhah G, Bentlage C, Björklund A (1995) Forelimb akinesia in the rat Parkinson model: differential effects of dopamine agonists and nigral transplants as assessed by a new stepping test. In: J Neurosci, pp 3863-3875.
Papa, S. M., and Chase, T. N. 1996. Levodopa-induced dyskinesias improved by a glutamate antagonist in Parkinsonian monkeys. Ann Neurol 39:574-578.
Schallert T, De Ryck M, Whishaw I Q, Ramirez V D, Teitelbaum P (1979) Excessive bracing reactions and their control by atropine and L-DOPA in an animal analog of Parkinsonism. In: Experimental Neurology, pp 33-43.
Stieger K, Belbellaa B, Le Guiner C, Moullier P, Rolling F (2009) In vivo gene regulation using tetracycline-regulatable systems. In: Adv Drug Deliv Rev, pp 527-541.
Zolotukhin S, Byrne B J, Mason E, Zolotukhin I, Potter M, Chesnut K, Summerford C, Samulski R J, Muzyczka N (1999) Recombinant adeno-associated virus purification using novel methods improves infectious titer and yield. In: Gene Ther, pp 973-985.

1. A gene expression system comprising:
a first nucleotide sequence encoding a fusion polypeptide of:
a) a destabilizing domain (DD), and
b) a GTPcyclohydrolase 1 (GCH1) polypeptide, or a biologically active fragment or variant thereof; and
a second nucleotide sequence encoding a tyrosine hydroxylase (TH) polypeptide, or a biologically active fragment or variant thereof.

2-4. (canceled)

5. A gene expression system according to claim 1, wherein said gene expression system comprises two vectors each containing one expression cassette, wherein:
the expression cassette in the first vector comprises the first nucleotide sequence and a first promoter sequence operably linked to the first nucleotide sequence, and
the expression cassette in the second vector comprises the second nucleotide and a second promoter sequence operably linked to the second nucleotide sequence.

6. A gene expression system according to claim 1, wherein said gene expression system comprises one vector comprising both the first nucleotide sequence and the second nucleotide sequence, wherein the vector comprises either:
i) one expression cassette, wherein
ia) a promotor is operably linked to either the first or the second nucleotide sequence, and wherein the nucleotide sequence to which the promotor is linked to the other of the first and second nucleotide sequence via a translation initiating nucleotide sequence, such as an internal ribosome entry site (IRES); or
ib) a promotor is operably linked to either the first or the second nucleotide sequence and wherein the nucleotide sequence to which the promotor is linked to the other of the first and the second nucleotide sequence via a 2A peptide; or
ii) two expression cassettes, wherein one expression cassette comprises the first nucleotide sequence and a first promoter sequence operably linked to first nucleotide sequence, and the other expression cassette comprises the second nucleotide and a second promoter sequence operably linked to the second nucleotide sequence; or
iii) a fusion polypeptide of the first nucleotide sequence and the second nucleotide sequence.

7-13. (canceled)

14. A method of treating a disease or condition associated with a reduced dopamine level comprising administering a gene expression system and a ligand binding to a destabilizing domain (DD) to a patient in need thereof, wherein said gene expression system comprises:
a first nucleotide sequence encoding a fusion polypeptide of:
a) a DD, and
b) a GTPcyclohydrolase 1 (GCH1) polypeptide, or a biologically active fragment or variant thereof; and
a second nucleotide sequence encoding a tyrosine hydroxylase (TH) polypeptide, or a biologically active fragment or variant thereof.

15. A method of treating a disease or condition associated with a reduced dopamine level in a patient that previously has been subject to gene therapy using a ligand binding to a destabilizing domain (DD), whereby a gene expression system comprising:
a first nucleotide sequence encoding a fusion polypeptide of:
a) a DD, and
b) a GTPcyclohydrolase 1 (GCH1) polypeptide, or a biologically active fragment or variant thereof; and
a second nucleotide sequence encoding a tyrosine hydroxylase (TH) polypeptide, or a biologically active fragment or variant thereof, has been administered to the brain of the patient.

16. The method of claim 15, wherein the treatment involved controlling the DOPA synthesis in the brain of the patient.

17. The method of claim 14, wherein said gene expression system comprises two vectors each containing one expression cassette, wherein:
the expression cassette in the first vector comprises the first nucleotide sequence and a first promoter sequence operably linked to the first nucleotide sequence, and
the expression cassette in the second vector comprises the second nucleotide and a second promoter sequence operably linked to the second nucleotide sequence.

18. The method of claim 15, wherein said gene expression system comprises two vectors each containing one expression cassette, wherein:
the expression cassette in the first vector comprises the first nucleotide sequence and a first promoter sequence operably linked to the first nucleotide sequence, and
the expression cassette in the second vector comprises the second nucleotide and a second promoter sequence operably linked to the second nucleotide sequence.

19. The method of claim 14, wherein said gene expression system comprises one vector comprising both the first nucleotide sequence and the second nucleotide sequence, wherein the vector comprises either:
i) one expression cassette, wherein
ia) a promotor is operably linked to either the first or the second nucleotide sequence, and wherein the nucleotide sequence to which the promotor is linked to the other of the first and second nucleotide sequence via a translation initiating nucleotide sequence, such as an internal ribosome entry site (IRES); or
ib) a promotor is operably linked to either the first or the second nucleotide sequence and wherein the nucleotide sequence to which the promotor is linked to the other of the first and the second nucleotide sequence via a 2A peptide; or
ii) two expression cassettes, wherein one expression cassette comprises the first nucleotide sequence and a first promoter sequence operably linked to first nucleotide sequence, and the other expression cassette comprises the second nucleotide and a second promoter sequence operably linked to the second nucleotide sequence; or
iii) a fusion polypeptide of the first nucleotide sequence and the second nucleotide sequence.

20. The method of claim 15, wherein said gene expression system comprises one vector comprising both the first nucleotide sequence and the second nucleotide sequence, wherein the vector comprises either:
i) one expression cassette, wherein
ia) a promotor is operably linked to either the first or the second nucleotide sequence, and wherein the nucleotide sequence to which the promotor is linked to the other of the first and second nucleotide sequence via a translation initiating nucleotide sequence, such as an internal ribosome entry site (IRES); or
ib) a promotor is operably linked to either the first or the second nucleotide sequence and wherein the nucleotide sequence to which the promotor is linked to the other of the first and the second nucleotide sequence via a 2A peptide; or
ii) two expression cassettes, wherein one expression cassette comprises the first nucleotide sequence and a first promoter sequence operably linked to first nucleotide sequence, and the other expression cassette comprises the second nucleotide and a second promoter sequence operably linked to the second nucleotide sequence; or
iii) a fusion polypeptide of the first nucleotide sequence and the second nucleotide sequence.

21. The method of claim 14, wherein said ligand binding to a DD is trimethoprim (TMP) or an analogue or derivative thereof.

22. The method of claim 15, wherein said ligand binding to a DD is trimethoprim (TMP) or an analogue or derivative thereof.

23. The method of claim 17, wherein said ligand binding to a DD is trimethoprim (TMP) or an analogue or derivative thereof.

24. The method of claim 18, wherein said ligand binding to a DD is trimethoprim (TMP) or an analogue or derivative thereof.

25. The method of claim 19, wherein said ligand binding to a DD is trimethoprim (TMP) or an analogue or derivative thereof.

26. The method of claim 20, wherein said ligand binding to a DD is trimethoprim (TMP) or an analogue or derivative thereof.

27. The method of claim 14, wherein said disease or condition is selected from the group consisting of idiopathic or genetic forms of Parkinson's disease, Parkinsonism and related disorders, schizophrenia, attention deficit disorder (ADD), attention deficit hyperactivity disorder (ADHD), autism spectrum disorders, and restless legs syndrome (RLS).

28. The method of claim 15, wherein said disease or condition is selected from the group consisting of idiopathic or genetic forms of Parkinson's disease, Parkinsonism and related disorders, schizophrenia, attention deficit disorder (ADD), attention deficit hyperactivity disorder (ADHD), autism spectrum disorders, and restless legs syndrome (RLS).

29. The method of claim 17, wherein said disease or condition is selected from the group consisting of idiopathic or genetic forms of Parkinson's disease, Parkinsonism and related disorders, schizophrenia, attention deficit disorder (ADD), attention deficit hyperactivity disorder (ADHD), autism spectrum disorders, and restless legs syndrome (RLS).

30. The method of claim 18, wherein said disease or condition is selected from the group consisting of idiopathic or genetic forms of Parkinson's disease, Parkinsonism and related disorders, schizophrenia, attention deficit disorder (ADD), attention deficit hyperactivity disorder (ADHD), autism spectrum disorders, and restless legs syndrome (RLS).

31. The method of claim 19, wherein said disease or condition is selected from the group consisting of idiopathic or genetic forms of Parkinson's disease, Parkinsonism and related disorders, schizophrenia, attention deficit disorder (ADD), attention deficit hyperactivity disorder (ADHD), autism spectrum disorders, and restless legs syndrome (RLS).

32. The method of claim 20, wherein said disease or condition is selected from the group consisting of idiopathic or genetic forms of Parkinson's disease, Parkinsonism and related disorders, schizophrenia, attention deficit disorder (ADD), attention deficit hyperactivity disorder (ADHD), autism spectrum disorders, and restless legs syndrome (RLS).

33. A gene expression system according to claim 1, wherein the destabilizing domain (DD) is based on DHFR.

34-35. (canceled)
